SEQUENCE LISTING (3 sequences)

SEQ ID NO: 1 - protein, 473 aa, Hepatitis C virus
MVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSHHHHHH

SEQ ID NO: 2 - protein, 9 aa, Hepatitis C virus
PTGSGKSTK

SEQ ID NO: 3 - protein, 8 aa, Artificial Sequence
  Description of Artificial Sequence conserved sequence motif VI wherein Xaa represents unconserved amino acids within the motif
QRXGRXGR

(19) United States
(12) Patent Application Publication
(10) Pub. No.: US 2004/0126809 A9
(43) Pub. Date: Jul. 1, 2004

(54) Hepatitis C virus helicase crystals, crystallographic structure and methods

(76) Inventors: Barry C. Finzel, Kalamazoo, MI (US); Melissa S. Harris, Marshall, MI (US); Eric T. Baldwin, Portage, MI (US)

Correspondence Address: MUETING, RAASCH & GEBHARDT, P.A., P.O. BOX 581415, MINNEAPOLIS, MN, 55458, US

(21) Appl. No.: 09/847,670

(22) Filed: May 2, 2001

Related U.S. Application Data
(60) Provisional application No. 60/201,598, filed on May 3, 2000.

(51) Int. Cl. G01N 33/53; C07K 1/00; C07K 14/00; C07K 17/00

(52) U.S. Cl. 435/7.1; 530/350

(57) ABSTRACT

Hepatitis C virus helicase has been crystallized as tetragonal and orthorhombic crystals, and the structures of the crystals has been solved. The structure coordinates of the crystal structures are useful for solving the structures of other molecules or molecular complexes.

This application claims the benefit of U.S. Provisional Application Serial No. 60/201,598, filed May 3, 2000, which is incorporated herein by reference in its entirety.

This application incorporates by reference the material contained on the duplicate (2) compact discs submitted herewith. Each disc contains the following files:

| Name | Size | Contents | Date of File Creation |
|---|---|---|---|
| table_1.txt | 214 KB | Table 1 | Apr. 25, 2001 |
| table_2.txt | 214 KB | Table 2 | Apr. 25, 2001 |
| table_3.txt | 203 KB | Table 3 | Apr. 25, 2001 |

### FIELD OF THE INVENTION

The invention relates to the crystallization and structure determination of Hepatitis C virus helicase.

### BACKGROUND OF THE INVENTION

The Hepatitis C virus (HCV) genome is translated as a large polyprotein of approximately 3000 amino acids that must be processed proteolytically to generate mature viral proteins, including coat (C) and envelope (E) proteins, and several non-structural (NS) enzymes necessary for viral replication (FIG. 1). Nonstructural protein three, NS3, is a bi-functional enzyme possessing both a serine protease activity, and an RNA helicase activity. Separate activities have been isolated on the N-terminal third and C-terminal two-thirds of the NS3 polypeptide. Both serine protease and helicase fragments have shown independent activity when expressed as isolated fragments in vitro, and both activities are required for viral replication, making this protein an attractive target for drug development.

HCV NS3 helicase is an NTP-dependent enzyme that unwinds duplex RNA and RNA:DNA hybrid substrates during viral replication. Several laboratories have reported structures of this enzyme in different crystal forms (Yao et al., Nat. Struct. Biol., 4: 463-77 (1997); Cho et al., J. Biol. Chem., 273:15045-52 (1998)), including one complex with bound single-stranded DNA (Kim et al., Structure, 6:89-100 (1998)). HCV NS3 helicase was found to include three domains. Two of these domains (d1 and d2) include homologous oligonucleotide-binding motifs conserved across helicase superfamilies (Korolev et al., Protein Science, 7:605-10 (1998)) that are thought to bind to individual phosphates along the backbone of the oligonucleotide substrate (Kim et al., Structure, 6:89-100 (1998)).

Although the above crystal structures have provided structural details for the enzyme in specific crystal forms, it would be desirable to have structures for additional crystal forms for comparison purposes. Such comparisons could be useful in helping to understand the protein structures by separating structural details that are merely a consequence of the environment of molecules in one crystal form from structural details that are independent of the crystal environment. Moreover, such comparisons might provide information applicable to better understanding the solution structure of the enzyme.

Multiple crystal forms can also be important for drug design processes. Structure-based drug design is dependent on the ability to produce crystalline complexes of enzyme and inhibitor, so that the interactions that make inhibitor-binding possible can be exploited in further chemical synthesis of analogs. While structures of native (uninhibited) enzyme are a necessary prerequisite to modeling studies, modeling alone can rarely predict correctly the bound geometry and orientation of even a very potent inhibitor. Weak inhibitors, such as preliminary lead compounds pose an even bigger problem for modeling. Structural data from analysis of a complex co-crystal is often the only way to probe molecular binding, and the only way to rationally move forward with a directed chemical analog program.

One common method for generating co-crystal structures is to soak an inhibitor into an existing native crystal form. However, problems frequently arise when intermolecular interactions that stabilize one particular crystal form are incompatible with ligand binding. The protein may be locked in a conformation that does not support binding, or the packing of protein molecules in the crystalline array may physically block access to a particular binding site. Alternatively, suitable solutions for growing protein crystals may not be optimized for inhibitor solubility. For example, the presence of salts in high concentrations may actually compete for inhibitor binding sites. These problems are sometimes alleviated by using alternate crystal forms.

### SUMMARY OF THE INVENTION

Two new crystal forms of Hepatitis C Virus NS3 helicase have been prepared. The crystal forms include a tetragonal form with two molecules in the crystallographic asymmetric unit (UHCV-A and UHCV-B), and an orthorhombic form (UHHO). Analysis of X-ray diffraction data from both forms confirms the overall three-domain structure of the enzyme reported by others in the study of different helicase crystal forms. The two new helicase structures differ from those previously reported in the packing relationship between molecules and in regard to the position of domain 2. Domain 2 is free to move in and out about a centrally located hinge, and different crystal forms trap the hinge motion in different conformational states. Comparison of the position of this domain in each of the available crystal structures reveals that the tetragonal form described herein represents the most closed conformational state of the hinge thusfar observed.

In one aspect, the present invention provides a molecule or molecular complex. In one embodiment, the molecule or molecular complex includes at least a portion of a Hepatitis C virus helicase or Hepatitis C virus helicase-like domain 1/domain 2 interface, wherein the domain 1/domain 2 interface includes amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively. In another embodiment, the molecule or molecular complex includes at least a portion of a Hepatitis C virus helicase or Hepatitis C virus helicase-like oligonucleotide binding site, wherein the oligonucleotide binding site includes amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively.

In another aspect, the present invention provides a Hepatitis C virus helicase molecule or molecular complex that includes at least a first and a second oligonucleotide binding site. In one embodiment, the distance between the first and the second oligonucleotide binding sites is less than about 21 angstroms. In another embodiment, the distance between the first and the second oligonucleotide binding sites is about 18.8 to about 19.5 angstroms.

In another aspect, the present invention provides a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3.

In another aspect, the present invention provides a scalable three-dimensional configuration of points, at least a portion of said points derived from structure coordinates of at least a portion of a Hepatitis C virus helicase molecule or molecular complex as listed in Tables 1, 2, or 3 and including at least one of a Hepatitis C virus helicase or Hepatitis C virus helicase-like domain 1/domain 2 interface, domain 1 oligonucleotide binding site, or domain 2 oligonucleotide binding site. Preferably, substantially all of the points are derived from structure coordinates of a Hepatitis C virus helicase molecule or molecular complex as listed in Tables 1, 2, or 3. Preferably, at least a portion of the points derived from the Hepatitis C virus helicase structure coordinates are derived from structure coordinates representing the locations of at least the backbone atoms of amino acids selected from the group consisting of (1) domain 1/domain 2 interface amino acids 205-209, 232-238, 415-420 and 460-467, (2) domain 1 oligonucleotide binding site amino acids 230-232, 255, 269, and 270-272, and (3) domain 2 oligonucleotide binding site amino acids 391-393, 411-413, 415, 416 and 460; as represented by structure coordinates of UHCV-A, UHCV-B, or UHHO in Tables 1, 2, and 3 respectively. The scalable three-dimensional configuration of points may optionally be displayed as a holographic image, a stereodiagram, a model or a computer-displayed image.

In another aspect, the present invention provides a scalable three-dimensional configuration of points, at least a portion of the points derived from structure coordinates of at least a portion of a molecule or a molecular complex that is structurally homologous to a Hepatitis C virus helicase molecule or molecular complex and includes at least one of a Hepatitis C virus helicase or Hepatitis C virus helicase-like domain 1/domain 2 interface, domain 1 oligonucleotide binding site, or domain 2 oligonucleotide binding site.

In another aspect, the present invention provides a machine-readable data storage medium including a data storage material encoded with machine readable data which, when using a machine programmed with instructions for using said data, is capable of displaying a graphical three-dimensional representation of at least one molecule or molecular complex selected from the group consisting of (i) a molecule or molecular complex including at least a portion of a Hepatitis C virus helicase or Hepatitis C virus helicase-like domain 1/domain 2 interface, wherein the domain 1/domain 2 interface includes amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively; (ii) a molecule or molecular complex including at least a portion of a Hepatitis C virus helicase or Hepatitis C virus helicase-like oligonucleotide binding site, wherein the oligonucleotide binding site includes amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively; (iii) a Hepatitis C virus helicase molecule or molecular complex including at least a first and a second oligonucleotide binding site, wherein the distance between the first and the second oligonucleotide binding sites is less than about 21 angstroms; and (iv) a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3.

In another aspect, the present invention provides a machine-readable data storage medium including a data storage material encoded with a first set of machine readable data which, when combined with a second set of machine readable data, using a machine programmed with instructions for using said first set of data and said second set of data, can determine at least a portion of the structure coordinates corresponding to the second set of machine readable data, wherein said first set of data includes a Fourier transform of at least a portion of the structure coordinates for Hepatitis C virus helicase listed in Tables 1, 2, or 3; and said second set of data includes an x-ray diffraction pattern of a molecule or molecular complex of unknown structure.

In another aspect, the present invention provides a method for obtaining structural information about a molecule or a molecular complex of unknown structure. The method includes crystallizing the molecule or molecular complex; generating an x-ray diffraction pattern from the crystallized molecule or molecular complex; and applying at least a portion of the structure coordinates set forth in Tables 1, 2, or 3 to the x-ray diffraction pattern to generate a three-dimensional electron density map of at least a portion of the molecule or molecular complex whose structure is unknown.

In another aspect, the present invention provides a method for homology modeling a Hepatitis C virus helicase homolog. The method includes aligning the amino acid sequence of a Hepatitis C virus helicase homolog with an amino acid sequence of Hepatitis C virus helicase (SEQ ID NO: 1) and incorporating the sequence of the Hepatitis C virus helicase homolog into a model of Hepatitis C virus helicase derived from structure coordinates set forth in Tables 1, 2, or 3 to yield a preliminary model of the Hepatitis C virus helicase homolog; subjecting the preliminary model to energy minimization to yield an energy minimized model; remodeling regions of the energy minimized model where stereochemistry restraints are violated to yield a final model of the Hepatitis C virus helicase homolog.

In anther aspect, the present invention provides a computer-assisted method for identifying, designing, and making inhibitors of Hepatitis C virus helicase activity. Preferably the invention provides compositions, more preferably pharmaceutical compositions, including such inhibotors.

In another aspect, the present invention provides a method for crystallizing a Hepatitis C virus helicase molecule or molecular complex including growing a crystal from a precipitant solution including purified Hepatitis C virus helicase, about 3% by weight to about 14% by weight PEG, about 5% by weight to about 15% by weight DMSO, and about 0.05M to about 0.07M potassium phosphate.

In another aspect, the present invention provides a method for co-crystallizing a Hepatitis C virus helicase molecule and a ligand to yield a molecular complex, including exchanging purified Hepatitis C virus helicase into a solution including HEPES, EDTA, and dithiothreitol; concentrating the Hepatitis C virus helicase to a concentration of about 12-16 mg/mL; combining concentrated Hepatitis C virus helicase with the ligand in a mixture including about 4% by weight to about 14% by weight PEG and about 5% by weight to about 15% by weight DMSO; and growing a co-crystal by vapor diffusion.

In another aspect, the present invention provides a method for crystallizing a Hepatitis C virus helicase molecule or molecular complex including growing a crystal by vapor diffusion with macro-seeding from a precipitant solution including purified Hepatitis C virus helicase, HEPES, and about 4% by weight to about 14% by weight mono-alkyl ether of PEG.

In another aspect, the present invention provides a method for co-crystallizing a Hepatitis C virus helicase molecule and a ligand to yield a molecular complex, including growing a crystal by vapor diffusion with macro-seeding from a precipitant solution including purified HCV helicase, HEPES, about 4% by weight to about 14% by weight mono-alkyl ether of PEG, and the ligand, wherein the ligand binds to at least one oligonucleotide binding site on the Hepatitis C virus helicase.

In another aspect, the present invention provides crystalline Hepatitis C virus helicase including a tetragonal crystal having unit cell dimensions of a=b=109 Å±3 Å; c=84 Å±2 Å; α=β=γ=90°; and space group P4₁; the unit cell containing two molecules in an asymmetric unit. Preferably, the invention provides a method for solving the structure of such crystals. Preferably, the invention provides methods for incorporating chemical entities in such crystals.

In another aspect, the present invention provides crystalline Hepatitis C virus helicase including an orthorhombic crystal characterized by unit cell dimensions of a=66 Å±2 Å; b=110 Å±3 Å; c=64 Å±2 Å; α=β=γ=90°; and a space group P2₁2₁2; the unit cell containing one molecule in the asymmetric unit. Preferably, the invention provides a method for solving the structure of such crystals. Preferably, the invention provides methods for incorporating chemical entities in such crystals.

Hepatitis C virus helicase crystals having orthorhombic crystal forms have been found to be surprisingly useful for incorporating chemical entities through crystal soaking methods. For example, the aqueous solubility of poorly soluble chemical entities is frequently enhanced by the addition of dimethylsulfoxide (DMSO) to the aqueous solution. The orthorhombic crystals of Hepatitis C virus helicase show unexpected stability upon immersion in such DMSO-containing aqueous solutions, resulting in the potential for increased effectiveness in the incorporation of chemical entities.

Hepatitis C virus helicase crystals having tetragonal crystal forms have also been found to be surprisingly useful for incorporating chemical entities through crystal soaking methods. For example, chemical entities which interact with the domain 1/domain 2 interface or span oligonucleotide binding sites in domain 1 and domain 2 may be incorporated through crystal soaking methods. The use of tetragonal crystals of Hepatitis C virus helicase in such soaking methods may lead to a better understanding of the binding interactions of Hepatitis C virus helicase with chemical entities.

Tables 1, 2, and 3 list the atomic structure coordinates for Hepatitis C virus helicase as derived by x-ray diffraction from crystals of UHCV-A, UHCV-B, and UHHO, respectively. Column 1 lists a number for the atom in the structure. Column 2 lists the element whose coordinates are measured. The first letter in the column defines the element. Column 3 lists the type of amino acid. Column 4 lists a number for the amino acid in the structure. Columns 5-7 list the crystallographic coordinates X, Y, and Z respectively. The crystallographic coordinates define the atomic position of the element measured. Column 8 lists an occupancy factor that refers to the fraction of the molecules in which each atom occupies the position specified by the coordinates. A value of “1” indicates that each atom has the same conformation, i.e., the same position, in all molecules of the crystal. Column 9 lists a thermal factor “B” that measures movement of the atom around its atomic center.

Abbreviations

The following abbreviations are used throughout this disclosure:

Hepatitis C virus (HCV)

Dimethyl sulfoxide (DMSO)

Polyethylene glycol (PEG)

Polyethylene glycol mono-methyl ether (PEGMME)

Dithiothreitol (DTT)

Multiple anomalous dispersion (MAD)

Root mean square (r.m.s.)

Root mean square deviation (r.m.s.d.)

The following abbreviations are used for amino acids throughout this disclosure:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

### BRIEF DESCRIPTION OF THE FIGURES

FIG. 1 is a schematic of the hepatitis C virus polyprotein organization; the C-terminal two-thirds of NS3 contains RNA helicase/NTPase activity.

FIG. 2 depicts the amino acid sequence of the HCV-1 genotype 1a construct used in this work; sequence motifs conserved (Gorbalenya et al., Current Opin. in Struct. Biol., 3:419-29 (1993)) in helicase are highlighted; secondary structure assignments are shown across the top, with alpha helices identified by solid bars and beta strands by hatched bars.

FIG. 3 is a ribbon drawing depicting the three-domain structure of the HCV helicase; the NTP-binding site is located on the surface of domain 1 that is closest to domain 2.

FIG. 4 shows a comparison of all the available crystal structures of HCV helicase with common domains 1 and 3 superimposed; different crystal structures show variability in the position of domain 2.

FIG. 5 shows a comparison of the positions of domain 2 for UHCV-A and the prior art structure 8OHM, which represent extremes of motion in domain 2 of available structures; common domains 1 and 3 have been overlaid. The motion of domain 2 can be envisioned as rotation about a hinge axis (solid black line). In view (a), the hinge axis (solid black line) is oriented vertically in the plane of the paper (similar to the orientation of FIG. 3); in view (b), the hinge axis (solid black line) is roughly perpendicular to the plane of the paper; in both (a) and (b), the V-shaped pair of vectors (solid black lines) illustrates the range of motion of centers of mass of the rotating domain (domain 2).

FIG. 6 shows a comparison of the conformation of the conserved NTP-binding loop (Walker motif A; residues 205-213; PTGSGKSTK; SEQ ID NO: 2) in the orthorhombic crystal form UHHO (a) and the tetragonal crystal form molecule A, UHCV-A (b); a single inorganic phosphate is bound at this site in UHHO.

### DETAILED DESCRIPTION OF THE INVENTION

Crystalline Forms of HCV Helicase and Method of Making

Applicants have produced crystals comprising HCV helicase which are suitable for X-ray crystallographic analysis. Thus, one embodiment of the invention provides a tetragonal crystal form of an HCV helicase characterized by unit cell dimensions of a=b=109 Å±3 Å; c=84 Å±2 Å, α=β=γ=90° and space group P4₁, with two molecules in the asymmetric unit. Another embodiment of the invention provides an orthorhombic crystal form of an HCV helicase contains characterized by unit cell dimensions of a=66 Å±2 Å; b=110 Å±3 Å; c=64 Å±2 Å, α=β=γ=90° and space group P2₁2₁2, with only one molecule in the asymmetric unit.

Accordingly, one aspect of the invention provides a Hepatitis C virus helicase or Hepatitis C virus helicase/ligand crystal. Native Hepatitis C virus helicase crystals may be prepared by methods described herein. In one embodiment, Hepatitis C virus helicase crystals may be grown from a precipitant solution including purified Hepatitis C virus helicase, about 3% by weight to about 14% by weight PEG, about 5% by weight to about 15% by weight DMSO, and about 0.05M to about 0.07M potassium phosphate. Preferably the DMSO concentration is about 7% by weight to about 12% by weight and PEG has a molecular weight of about 2,000 to 20,000 daltons. In another embodiment, Hepatitis C virus helicase crystals may be grown by vapor diffusion with macro-seeding from a precipitant solution comprising purified Hepatitis C virus helicase, HEPES, and about 4% by weight to about 14% by weight mono-alkyl ether of PEG. Preferably the mono-alkyl ether of PEG is a mono-methyl ether of PEG. Preferably the PEG has a molecular weight of about 2,000 to 20,000 daltons.

In addition, Hepatitis C virus helicase/ligand crystals may be prepared by methods including soaking existing native crystals in a solution containing the ligand, or by growing crystals under conditions similar to the crystallization conditions for the native crystals but in the presence of chemical entities. Variation in buffer and buffer pH as well as other additives such as PEG is apparent to those skilled in the art and may result in similar crystals.

X-ray Crystallographic Analysis

Using high resolution X-ray crystallography, the three-dimensional structures of two unique crystal forms of the HCV helicase (genotype-1, strain 1A) have been solved. These new crystal forms are identified herein as UHCV and UHHO. The constituent amino acids of both UHCV and UHHO are defined by a set of structure coordinates as set forth in Tables 1, 2, and 3. The term “structure coordinates” refers to Cartesian coordinates derived from mathematical equations related to the patterns obtained on diffraction of a monochromatic beam of x-rays by the atoms (scattering centers) of an HCV helicase in crystal form. The diffraction data are used to calculate an electron density map of the repeating unit of the crystal. The electron density maps are then used to establish the positions of the individual atoms of the HCV helicase protein or protein/ligand complex.

It will be understood by one of skill in the art that slight variations in structure coordinates can be generated by mathematically manipulating the HCV helicase structure coordinates. For example, the structure coordinates set forth in Tables 1, 2, and 3 could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be “structurally equivalent.” Structural equivalence is described in more detail below.

It should be noted that slight variations in individual structure coordinates of the HCV helicase, as defined above, would not be expected to significantly alter the nature of the chemical entities that could associate with the binding sites. Thus, for example, a ligand that bound to an oligonucleotide binding site of HCV helicase would also be expected to bind to another binding site whose structure coordinates define a shape that falls within the acceptable error.

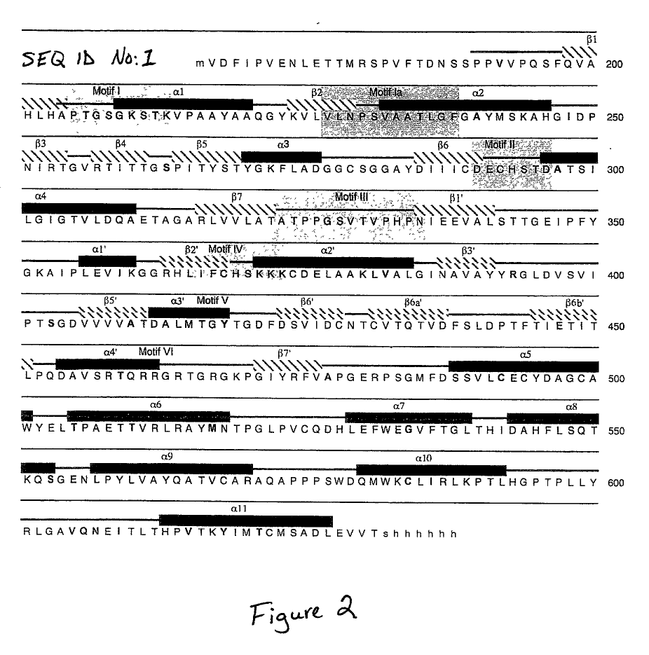

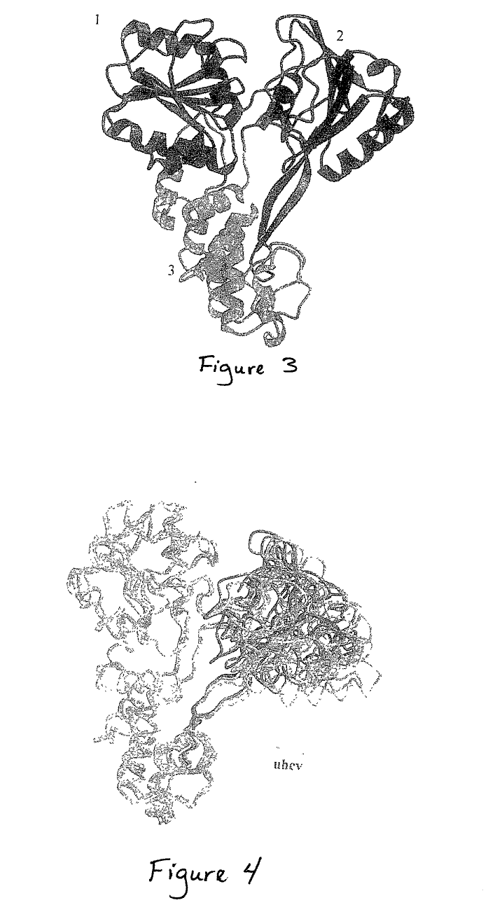

Both crystal forms of HCV helicase (UHCV and UHHO) arise from crystallization trials with a helicase fragment consisting of NS3 residues 166-631 as defined in FIG. 2, (polyprotein residues 1192-1657) (Jin et al., Arch. Biochem. Biophys., 323:47-53 (1995)) including a C-terminal SH₆-tag to facilitate purification from a recombinant system. A tetragonal crystal form and an orthorhombic crystal form were characterized at high resolution. The tetragonal form, characterized by unit cell a=b=109 Å, c=84 Å and space group P4₁, has two molecules in the asymmetric unit. This structure has been solved by molecular replacement and refined at 2.0 Å resolution (R=0.228). The orthorhombic form, with intermolecular interactions unrelated to the tetragonal form, is orthorhombic, space group P2₁2₁2 (a=66 Å, b=110 Å, c=64 Å), with only one molecule in the asymmetric unit. This structure has been refined against 1.8 Ådata (R=0.206). Both structures confirm the overall structural topology previously described by others. Secondary structure assignments are summarized in FIG. 2. The enzyme has a structure composed of three domains, each of roughly 140 amino acids (FIG. 3). Domains 1 and 2 share a similar alpha-beta fold with a centralized parallel beta sheet surrounded by helices. Domain 3 adopts a different alpha-helical fold.

The two crystal structure determinations result in three crystallographically independent observations of the helicase enzyme structure (two from the tetragonal form crystals and one from the orthorhombic form). These structures, identified herein as UHCV-A, UHCV-B (tetragonal form molecules A and B) and UHHO (orthorhombic form), can be compared to helicase structures available from analysis of other crystal forms 1HEI-A, 1HEI-B, 1A1V and 8OHM (See Table 6 in Example III for identification and references). These structures have been superimposed in a variety of different ways, and the r.m.s. differences in structure are summarized in Table 6. Differences in all Cα positions after superposition are 0.89 Å (UHCV-A vs. UHCV-B), and 1.52 Å (UHCV-A vs. UHHO). The overall structures of individual domains (e.g., domain 3 vs. domain 3, or domain 2 vs. domain 2) are closely conserved in all the crystal forms.

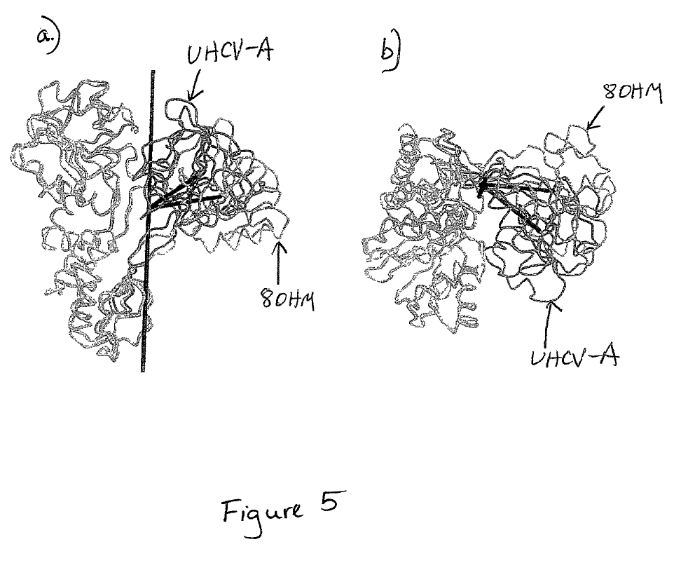

There is an extensive and rigid interface between domains 1 and 3 formed by hydrophobic complementarity of domain 1 helix α4 with domain 3 helices α5 and α6; these two domains have the same fixed relationship to each other in all reported crystal structures. (Compare r.m.s. differences in position of Table 6 based on superimpositions with combined d1/d3 to those of d1 or d3 individually). In contrast, domain 2 is only loosely associated with domain 1, and the interface to domain 3 is limited to contacts on the extreme end of a long beta hairpin that extends downward to lie against the back of domain 3 (FIG. 3). Significant variation in the position of domain 2 has been reported with respect to the fixed domains 1 and 3, and this has been likened to rotation of the domain about a centrally located hinge (Cho et al., J. Biol. Chem., 273:15045-52 (1998)). This rotation is readily apparent when all domains 1 and 3 are superimposed (FIG. 4).

The most striking difference in enzyme structure observed upon comparison of these new crystal forms with known crystal forms is the position of domain 2 with respect to domain 1 about the centrally located hinge. Without intending to be bound by theory or mechanism, it is believed that the in-and-out movement of domain 2 relative to fixed domains 1 and 3 may play a role in deforming and translocating oligonucleotide substrates during catalysis. Conserved sequence motifs in domain 1 define the site of NTP binding and hydrolysis near the d1/d2 interface, so reaction with the NTP co-factor may be necessary to facilitate this conformational change.

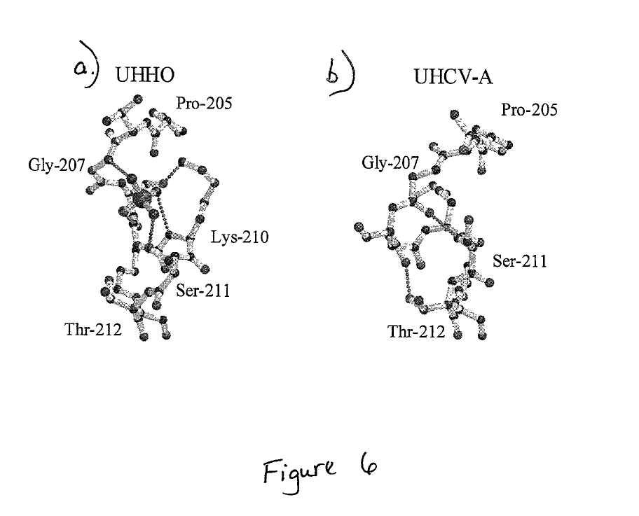

The rotation of domain 2 around the centrally located hinge was quantitated for five known helicase crystal forms using the analytical method of Wriggers & Schulten (Proteins: Structure, Function, and Genetics, 29:1-14 (1997)) (Table 7). Motion of domain 2 can be described as rotation around a vertical hinge axis near residue 484. Data for UHCV-B is not shown since, by this analysis, the angle of rotation differentiating UHCV-A and UHCV-B is small (2.8°). A similar rotation angle was noted by Yao et al. (Nat. Struct. Biol., 4: 463-77 (1997)) in their comparison of the two molecules in their crystal form (1HEI-A vs 1HEI-B); these trivial comparisons have been omitted from the tabulation. The tetragonal crystal form UHCV-A serves as a convenient reference state, because it represents the most closed state of the hinge. 8OHM, with domain 2 rotated 32.3°, represents the opposite extreme (FIG. 5). Models 1HEI and 1A1V are similar to each other, and represent intermediate hinge states of 14.2° and 9.5° rotations from UHCV-A, respectively. Together, these structures appear to define a continuum of domain rotations about the same vertical hinge axis. As a consequence of these movements, the distance between the oligonucleotide binding sites changes (Table 7).

The hinge motion impacts the enzyme structure at two important catalytic sites, the NTP-binding site and the oligonucleotide-binding sites. The NTP-binding site, identified by the conserved tri-phosphate binding Motif I (Walker Motif “A”; Walker et al., EMBO J., 1:945-51 (1982)) lies on the surface of domain 1 facing the broad gap between domains 1 and 2. While the gap between domains is not closed in any of the available crystal forms (it is smallest in UHCV-A), one can imagine an extreme hinge conformation, perhaps induced by binding or hydrolysis of ATP in domain 1, that brings domain 2 into direct contact with domain 1. Conserved sequence motif VI (residues Q₄₆₀RxGRxGR467 where x represents unconserved amino acids within the motif; SEQ ID NO: 3) on domain 2 has been suggested as an important mediator of this allosteric effect (Kim et al., Structure, 6:89-100 (1998)).

Based on the structural analysis of a helicase/single-stranded DNA co-crystal, Kim et al. (Structure, 6:89-100 (1998)) have identified oligonucleotide binding sites on both domains 1 and 2. The distance between these two sites (defined by residues G₂₅₅ and T₂₆₉ in d1, and R₃₉₃ and T₄₁₁ in d2), changes by as much as 8 Å when domain 2 swings from one extreme state (e.g., UHCV-A) to the other (8OHM). A single oligonucleotide held at both sites must either undergo a large conformational change or dissociate (and re-associate) at one end or the other as the enzyme undergoes a hinge-state change. Either action might be utilized effectively during processive substrate strand separation.

Because the hinge domain (domain 2) of the helicase is further toward a closed position in the tetragonal form structures UHCV-A and UHCV-B than in any other crystal structure reported to date, this crystal form is particularly well-suited for the characterization of inhibitors that span the two oligonucleotide-binding sites of the enzyme, or compounds that interfere with allosteric motions of domain 2 by binding across the d1/d2 interface. It may be less well suited for studying inhibitors that bind at the NTP-binding site, as access to this site is blocked in one of the two molecules in the asymmetric unit by intermolecular contacts introduced from crystal symmetry.

The orthorhombic form of UHHO represents a conformational intermediate that is not easily modeled as a hinge, but appears as a conformation intermediate between the extreme positions of domain 2. The fact that the orthorhombic crystal form UHHO has an accessible NTP-binding site occupied by inorganic phosphate in the native crystal form suggests that it may be uniquely suited for the study of inhibitors or co-factors that bind at the NTP-binding site. This crystal form is also desirable in the study of cofactors becasue it grows from solutions that contain significant quantities of DMSO, which is often required to bring marginally soluble chemical entities into solution with HCV helicase.

Beyond the domain 2 position, notable structural differences in the HCV helicase crystal forms are isolated in only a few areas. The NTP-binding loop (conserved Motif I; residues 205-214) appears to be quite flexible, and the exact conformation of this loop varies from structure to structure. The presence or absence of bound cation co-factor (Mg⁺²), or counter-anion (SO₄⁻² or PO₄⁻²) affects the geometry, as does the crystal environment. The structure of this loop in the orthorhombic form UHHO may be most representative of the active enzyme. In this structure, each of the four oxygen atoms of the phosphate ion is bound and held through hydrogen bonds to backbone amides of G207, G209 and K210, Oγ of S211 and Nz of K210 (FIG. 6a). This inorganic phosphate occupies a position taken by the beta-phosphate of nucleoside tri-phosphates in other NTP-binding enzymes that share this sequence motif (Pai et al., EMBO J., 9:2351 (1990); Tari et al., Nat. Struct. Biol., 3:355 (1996)). In contrast to molecule A of the tetragonal form (UHCV-A), this loop adopts a different geometry (FIG. 6b), in which the loop is collapsed on itself, making only intramolecular H-bonds. This presumably inactive conformation is apparently stabilized through contacts with a crystallographically related molecule. In UHCV-B, the loop adopts a geometry more similar to the consensus geometry represented by UHHO.

Conformational States and Binding Sites

Applicants' invention has provided, for the first time, information about extent of the relative motion of domain 2 relative to domain 1 in HCV helicase. In particular, the identification of a substantially “closed” conformation, represented by the UHCV structure, that affects access to the NTP binding site on domain 1 and alters the relative locations of the oligonucleotide binding sites on domains 1 and 2 has far reaching ramifications for drug discovery and design.

It is well known that structural information about protein binding sites is of significant utility in fields such as drug discovery. The association of natural ligands, substrates, cofactors and the like with binding sites on enzymes or receptors is the basis of many biological mechanisms of action. Similarly, many drugs exert their biological effects through association with the binding sites of receptors and enzymes. Such associations may occur with all or any parts of the binding site. An understanding of such associations helps lead to the design of drugs having more favorable associations with their target, and thus improved biological effects. Therefore, this information is valuable in designing potential inhibitors of HCV helicase-like binding sites, as discussed in more detail below.

The term “binding site” as used herein refers to a region of a molecule or molecular complex, that, as a result of its shape, favorably associates with another chemical entity. A “chemical entity,” as that term is used herein, includes chemical compounds, complexes of two or more chemical compounds, and fragments of such compounds or complexes. Chemical entities that are determined to associate with HCV helicase are potential drug candidates. The term “HCV helicase-like binding site” refers to a portion of a molecule or molecular complex whose shape is sufficiently similar to at least a portion of a binding site of HCV helicase as to be expected to bind related ligands. The term “associating with” refers to a condition of proximity between a chemical entity, or portions thereof, and an HCV helicase molecule or portions thereof. The association may be non-covalent, wherein the juxtaposition is energetically favored by hydrogen bonding, van der Waals forces, or electrostatic interactions, or it may be covalent.

In the present invention, four different binding sites for HCV helicase are identified. First, an NTP binding site is present on the surface of domain 1 of HCV helicase. As noted above, the NTP-binding site is identified by the conserved tri-phosphate binding Motif I (Walker Motif “A”; Walker et al., EMBO J., 1:945-51 (1982)) that lies on the surface of domain 1 facing the broad gap between domains 1 and 2. HCV helicase also includes two oligonucleotide binding sites, one on domain 1 and the other on domain 2. The two oligonucleotide sites can also be defined in relation to each other. The distance (defined as the distance between the side chain oxygen of T₂₆₉ and the side chain oxygen of T₄₁₁) between these sites (defined by residues G₂₅₅ and T₂₆₉ in domain 1, and R₃₉₃ and T₄₁₁ in domain 2) changes by as much as 8 Å when domain 2 swings from one extreme state (e.g., UHCV-A) to the other (8OHM). The distance between the two oligonucleotide binding sites preferably is less than about 21 angstroms and more preferably is about 18.8 to about 19.5 angstroms.

HCV helicase also possesses an allosteric binding site at the interface between domains 1 and 2. Conserved sequence motif VI (residues Q₄₆₀RxGRxGR₄₆₇ where x represents unconserved amino acids within the motif; SEQ ID NO: 3) on domain 2 has been suggested as an important mediator of this allosteric effect (Kim et al., Structure, 6:89-100 (1998)).

In one aspect, the binding sites of HCV helicase include the set of structure coordinates of all atoms in their respective constituent amino acids; in another aspect, the binding sites include the set of structure coordinates of just the backbone atoms of their respective constituent atoms. It will be readily apparent to those of skill in the art that the numbering of amino acids in other isoforms of HCV helicase may be different than that of other HCV helicase isoforms.

Three-Dimensional Configurations

The structure coordinates listed in Tables 1, 2, or 3 for the tetragonal and orthorhombic crystal forms of HCV helicase, or for a domain thereof or a portion of a domain, such as for one of the binding sites of HCV helicase, define a unique, scalable configuration of points in space. Those of skill in the art understand that a set of structure coordinates for protein or an protein/ligand complex, or a portion thereof, defines a relative set of spatially distributed points that, in turn, defines a configuration in three dimensions. A similar or identical configuration can be defined by an entirely different set of coordinates, provided the distances and angles between the points defined by the coordinates remain essentially the same. It will be further understood that this three dimensional configuration is scalable; that is, smaller and larger configurations are uniquely defined by the relative distances between and among the points, and the angles defined by any three points.

The present invention thus includes a scalable three-dimensional configuration of points defined by the structure coordinates of at least a portion of an HCV helicase molecule, as shown in Tables 1, 2, or 3, as well as structurally equivalent configurations, as described below. A “scalable three-dimensional configuration” that is defined by a set of structure coordinates includes not just the particular configuration defined by the set of structure coordinates but also those scaled configurations defined by the relative distances between and among the points defined by the structure coordinates, and the angles defined by any three points. It will be understood that slight variations in the positions of one or more points will not substantially alter the three-dimensional configuration defined by a set of structure coordinates, and configurations including such slight variations are included in this embodiment of the invention. Such a slight variation in position is preferably less than about 1.5A, more preferably less than about 1.0 Å, between the point with the varied position and the point nearest to it.

Preferably, the three-dimensional configuration includes points defined by structure coordinates representing the locations of a plurality of the amino acids defining an HCV helicase binding site. More preferably, the three dimensional configuration includes points defined by structure coordinates representing locations of a plurality of amino acids defining domain 2 of HCV helicase and, optionally, at least a portion of domain 1. In one aspect, the three-dimensional configuration includes points defined by structure coordinates representing the locations of just the backbone atoms of the plurality of amino acids. Preferably, the backbone atoms include the backbone atoms of amino acids selected from the group consisting of (1) domain 1/domain 2 interface amino acids 205-209, 232-238, 415-420 and 460-467, (2) domain 1 oligonucleotide binding site amino acids 230-232, 255, 269, and 270-272, and (3) domain 2 oligonucleotide binding site amino acids 391-393, 411-413, 415, 416 and 460; in another aspect, the three-dimensional configuration includes points defined by structure coordinates representing the locations of the side chain and the backbone atoms (other than hydrogens) of the plurality of amino acids. Preferably, the side chain and backbone atoms include the side chain and backbone atoms amino acids selected from the group consisting of (1) domain 1/domain 2 interface amino acids 205-209, 232-238, 415-420 and 460-467, (2) domain 1 oligonucleotide binding site amino acids 230-232, 255, 269, and 270-272, and (3) domain 2 oligonucleotide binding site amino acids 391-393, 411-413, 415, 416 and 460. In yet another aspect, the three-dimensional configuration includes points defined by structure coordinates representing the locations the backbone atoms of at least 30 amino acids that are contiguous in the amino acid sequence of HCV helicase (SEQ ID NO: 1). In still another aspect, the three-dimensional configuration includes points defined by structure coordinates representing the locations the side chain atoms and the backbone atoms of at least 30 amino acids that are contiguous in the amino acid sequence of HCV helicase (SEQ ID NO: 1).

Likewise, the invention also includes a three-dimensional configuration of points defined by structure coordinates of molecules or molecular complexes that are structurally homologous to HCV helicase, as well as structurally equivalent configurations. Structurally homologous molecules or molecular complexes are defined below. Advantageously, structurally homologous molecules can be identified using the structure coordinates of HCV helicase (Tables 1, 2, and 3) according to a method of the invention.

The configurations of points in space defined by structure coordinates according to the invention can be visualized as, for example, a holographic image, a stereodiagram, a model or a computer-displayed image, and the invention thus includes such images, diagrams or models.

Structural Equivalence

“Structural equivalence,” as the term is used herein, describes a relationship between the three-dimensional structures of two molecules or portions thereof, e.g., two crystal structures. Various computational analyses can be used to determine whether a molecule or portion thereof is “structurally equivalent” to all or part of an HCV helicase such as UHCV-A, UHCV-B, or UHHO represented by the structure coordinates in Tables 1, 2, or 3. Such analyses may be carried out in current software applications, such as the Molecular Similarity application of QUANTA (Molecular Simulations Inc., San Diego, Calif.) version 4. 1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or binding site thereof, or any portion thereof, that has a root mean square deviation of conserved residue backbone atoms (N, Cα, C, O) of less than 1.5 Å, when superimposed on the relevant backbone atoms described by the reference structure coordinates listed in Tables 1, 2, or 3, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates in Tables 1, 2, or 3, “a root mean square deviation from the conserved backbone atoms of those amino acids of not more than 1.5 Å. More preferably, the root mean square deviation is less than about 1.0 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of HCV helicase or a binding site portion thereof, as defined by the structure coordinates of HCV helicase described herein.

Machine Readable Storage Media

Transformation of the structure coordinates for all or a portion of Hepatitis C virus helicase or the Hepatitis C virus helicase/ligand complex or one of its binding sites, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium comprising a data storage material encoded with machine readable data which, when using a machine programmed with instructions for using said data, is capable of displaying a graphical three-dimensional representation of any of the molecule or molecular complexes of this invention that have been described above. In a preferred embodiment, the machine-readable data storage medium comprises a data storage material encoded with machine readable data which, when using a machine programmed with instructions for using said data, is capable of displaying a graphical three-dimensional representation of a molecule or molecular complex comprising all or any parts of a Hepatitis C virus helicase binding site or a Hepatitis C virus helicase-like binding site, as defined above. In another preferred embodiment, the machine-readable data storage medium is capable of displaying a graphical three-dimensional representation of a molecule or molecular complex defined by the structure coordinates of all of the amino acids in Tables 1, 2, or 3,±a root mean square deviation from the backbone atoms of said amino acids of not more than 1.5 Å.

In an alternative embodiment, the machine-readable data storage medium comprises a data storage material encoded with a first set of machine readable data which comprises the Fourier transform of the structure coordinates set forth in Tables 1, 2, or 3, and which, when using a machine programmed with instructions for using said data, can be combined with a second set of machine readable data comprising the x-ray diffraction pattern of a molecule or molecular complex to determine at least a portion of the structure coordinates corresponding to the second set of machine readable data.

For example, a system for reading a data storage medium may include a computer comprising a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid cyrstal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may comprise CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

Structurally Homologous Molecules, Molecular Complexes, and Crystal Structures

The structure coordinates set forth in Tables 1, 2, or 3 can be used to aid in obtaining structural information about another crystallized molecule or molecular complex. A “molecular complex” means a protein in covalent or non-covalent association with a chemical entity or compound. The method of the invention allows determination of at least a portion of the three-dimensional structure of molecules or molecular complexes which contain one or more structural features that are similar to structural features of Hepatitis C virus helicase. These molecules are referred to herein as “structurally homologous” to Hepatitis C virus helicase. Similar structural features can include, for example, regions of amino acid identity, conserved active site or binding site motifs, and similarly arranged secondary structural elements (e.g., α helices and β sheets) and the assembly of these elements into domains. Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al., FEMS Microbiol Lett., 174:247-50 (1999), and available at http://www.ncbi.nlm.nih.gov/gorf/b12.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” Preferably, a structurally homologous molecule is a protein that has an amino acid sequence sharing at least 65% identity with the amino acid sequence of Hepatitis C virus helicase (SEQ ID NO: 1). More preferably, a protein that is structurally homologous to Hepatitis C virus helicase includes at least one contiguous stretch of at least 50 amino acids that shares at least 80% amino acid sequence identity with the analogous portion of Hepatitis C virus helicase. Methods for generating structural information about the structurally homologous molecule or molecular complex are well-known and include, for example, molecular replacement techniques.

Therefore, in another embodiment this invention provides a method of utilizing molecular replacement to obtain structural information about a molecule or molecular complex whose structure is unknown comprising the steps of:

(a) crystallizing the molecule or molecular complex of unknown structure;

(b) generating an x-ray diffraction pattern from said crystallized molecule or molecular complex; and

(c) applying at least a portion of the structure coordinates set forth in Tables 1, 2, or 3 to the x-ray diffraction pattern to generate a three-dimensional electron density map of the molecule or molecular complex whose structure is unknown.

By using molecular replacement, all or part of the structure coordinates of Hepatitis C virus helicase or the Hepatitis C virus helicase/ligand complex as provided by this invention (and set forth in Tables 1, 2, or 3) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of Hepatitis C virus helicase or the Hepatitis C virus helicase/ligand complex according to Tables 1, 2, or 3 within the unit cell of the crystal of the unknown molecule or molecular complex so as best to account for the observed x-ray diffraction pattern of the crystal of the molecule or molecular complex whose structure is unknown. Phases can then be calculated from this model and combined with the observed x-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions,” in Meth. Enzymol., 115:55-77 (1985); M. G. Rossman, ed., “The Molecular Replacement Method,” Int. Sci. Rev. Ser., No. 13, Gordon & Breach, New York (1972)).

Structural information about a portion of any crystallized molecule or molecular complex that is sufficiently structurally homologous to a portion of Hepatitis C virus helicase can be resolved by this method. In addition to a molecule that shares one or more structural features with Hepatitis C virus helicase as described above, a molecule that has similar bioactivity, such as the same catalytic activity, substrate specificity or ligand binding activity as Hepatitis C virus helicase, may also be sufficiently structurally homologous to Hepatitis C virus helicase to permit use of the structure coordinates of Hepatitis C virus helicase to solve its crystal structure.

In a preferred embodiment, the method of molecular replacement is utilized to obtain structural information about a molecule or molecular complex, wherein the molecule or molecular complex comprises at least one Hepatitis C virus helicase subunit or homolog. A “subunit” of Hepatitis C virus helicase is a Hepatitis C virus helicase molecule that has been truncated at the N-terminus or the C-terminus, or both. In the context of the present invention, a “homolog” of Hepatitis C virus helicase is a protein that contains one or more amino acid substitutions, deletions, additions, or rearrangements with respect to the amino acid sequence of Hepatitis C virus helicase, but that, when folded into its native conformation, exhibits or is reasonably expected to exhibit at least a portion of the tertiary (three-dimensional) structure of Hepatitis C virus helicase. For example, structurally homologous molecules can contain deletions or additions of one or more contiguous or noncontiguous amino acids, such as a loop or a domain. Structurally homologous molecules also include “modified” Hepatitis C virus helicase molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

A heavy atom derivative of Hepatitis C virus helicase is also included as a Hepatitis C virus helicase homolog. The term “heavy atom derivative” refers to derivatives of Hepatitis C virus helicase produced by chemically modifying a crystal of Hepatitis C virus helicase. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by x-ray diffraction analysis of the soaked crystal. This information, in turn, is used to generate the phase information used to construct three-dimensional structure of the protein (T. L. Blundell and N. L. Johnson, Protein Crystallography, Academic Press (1976)).

Because Hepatitis C virus helicase can crystallize in more than one crystal form, the structure coordinates of Hepatitis C virus helicase as provided by this invention are particularly useful in solving the structure of other crystal forms of Hepatitis C virus helicase or Hepatitis C virus helicase complexes.

The structure coordinates of HCV helicase as provided by this invention are particularly useful in solving the structure of HCV helicase mutants. Mutants may be prepared, for example, by expression of HCV helicase cDNA previously altered in its coding sequence by oligonucleotide-directed mutagenesis. Mutants may also be generated by site-specific incorporation of unnatural amino acids into HCV helicase proteins using the general biosynthetic method of C. J. Noren et al., Science, 244:182-188 (1989). In this method, the codon encoding the amino acid of interest in wild-type HCV helicase is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant HCV helicase with the site-specific incorporated unnatural amino acid.

Selenocysteine or selenomethionine may be incorporated into wild-type or mutant HCV helicase by expression of HCV helicase-encoding cDNAs in auxotrophic E. coli strains (Hendrickson et al., EMBO J., 9(5): 1665-72 (1990)). In this method, the wild-type or mutagenized HCV helicase cDNA may be expressed in a host organism on a growth medium depleted of either natural cysteine or methionine (or both) but enriched in selenocysteine or selenomethionine (or both).

The structure coordinates of Hepatitis C virus helicase in Tables 1, 2, or 3 are also particularly useful to solve the structure of crystals of Hepatitis C virus helicase, Hepatitis C virus helicase mutants or Hepatitis C virus helicase homologs co-complexed with a variety of chemical entities. This approach enables the determination of the optimal sites for interaction between chemical entities, including candidate Hepatitis C virus helicase inhibitors and Hepatitis C virus helicase. Potential sites for modification within the various binding site of the molecule can also be identified. This information provides an additional tool for determining the most efficient binding interactions, for example, increased hydrophobic interactions, between Hepatitis C virus helicase and a chemical entity.

For example, high resolution x-ray diffraction data collected from crystals exposed to different types of solvent allows the determination of where each type of solvent molecule resides. Small molecules that bind tightly to those sites can then be designed and synthesized and tested for their Hepatitis C virus helicase inhibition activity.

All of the complexes referred to above may be studied using well-known x-ray diffraction techniques and may be refined versus 1.5-3 Å resolution x-ray data to an R value of about 0.20 or less using computer software, such as X-PLOR (Yale University, 1992, distributed by Molecular Simulations, Inc.; see, e.g., Blundell & Johnson, supra; Meth. Enzymol., Vol. 114 & 115, H. W. Wyckoff et al., eds., Academic Press (1985)). This information may thus be used to optimize known Hepatitis C virus helicase inhibitors, and more importantly, to design new Hepatitis C virus helicase inhibitors.

The invention also includes the unique three-dimensional configuration defined by a set of points defined by the structure coordinates for a molecule or molecular complex structurally homologous to Hepatitis C virus helicase as determined using the method of the present invention, structurally equivalent configurations, and magnetic storage media comprising such set of structure coordinates.

Further, the invention includes structurally homologous molecules as identified using the method of the invention.

Homology Modeling

Using homology modeling, a computer model of a Hepatitis C virus helicase homolog can be built or refined without crystallizing the homolog. First, a preliminary model of the Hepatitis C virus helicase homolog is created by sequence alignment with Hepatitis C virus helicase, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. Where the Hepatitis C virus helicase homolog has been crystallized, the final homology model can be used to solve the crystal structure of the homolog by molecular replacement, as described above. Next, the preliminary model is subjected to energy minimization to yield an energy minimized model. The energy minimized model may contain regions where stereochemistry restraints are violated, in which case such regions are remodeled to obtain a final homology model. The homology model is positioned according to the results of molecular replacement, and subjected to further refinement comprising molecular dynamics calculations.

Rational Drug Design

Computational techniques can be used to screen, identify, select and design chemical entities capable of associating with Hepatitis C virus helicase or structurally homologous molecules. Such ligands can include, for example, (a) inhibitors of HCV helicase that bind to at least one of the oligonucleotide binding sites of HCV helicase; (b) compounds that interfere with the allosteric motion of domain 2 of HCV helicase by binding at the interface between domain 1 and domain 2 of HCV helicase; and (c) inhibitors or cofactors that bind to the NTP binding site on domain 1 of HCV helicase. Computational techniques can be used to screen, identify, select and design chemical entities capable of associating with HCV helicase or structurally homologous molecules. Knowledge of the structure coordinates for the two new crystal forms of HCV helicase permits the design and/or identification of natural of synthetic compounds that have a shape complementary to the conformation of one or more of the four HCV helicase binding sites identified herein. In particular, computational techniques can be used to identify or design chemical entities, such as inhibitors, cofactors, allosteric effectors, agonists and antagonists, that associate with an HCV helicase binding site or an HCV helicase-like binding site. Inhibitors may bind to all or a portion of a binding site of HCV helicase, and can be competitive, non-competitive, or uncompetitive inhibitors; or interfere with dimerization by binding at the interface between the two monomers. Once identified and screened for biological activity, these chemical entities may be used therapeutically or prophylactically to block HCV helicase activity and, thus, to treat Hepatitis C virus infection. Structure-activity data for analogs of ligands bind to HCV helicase or HCV helicase-like binding sites can also be obtained computationally.

The term “chemical entity,” as used herein, refers to chemical compounds, complexes of two or more chemical compounds, and fragments of such compounds or complexes. Chemical entities that are determined to associate with Hepatitis C virus helicase are potential drug candidates. Data stored in a machine-readable storage medium that is capable of displaying a graphical three-dimensional representation of the structure of Hepatitis C virus helicase or a structurally homologous molecule, as identified herein, or portions thereof may thus be advantageously used for drug discovery. The structure coordinates of the chemical entity are used to generate a three-dimensional image that can be computationally fit to the three-dimensional image of Hepatitis C virus helicase or a structurally homologous molecule. The three-dimensional molecular structure encoded by the data in the data storage medium can then be computationally evaluated for its ability to associate with chemical entities. When the molecular structures encoded by the data is displayed in a graphical three-dimensional representation on a computer screen, the protein structure can also be visually inspected for potential association with chemical entities.

One embodiment of the method of drug design involves evaluating the potential association of a known chemical entity with Hepatitis C virus helicase or a structurally homologous molecule, particularly with a Hepatitis C virus helicase binding site or Hepatitis C virus helicase-like binding site. The method of drug design thus includes computationally evaluating the potential of a selected chemical entity to associate with any of the molecules or molecular complexes set forth above. This method comprises the steps of: (a) employing computational means to perform a fitting operation between the selected chemical entity and a binding site of the molecule or molecular complex; and (b) analyzing the results of said fitting operation to quantify the association between the chemical entity and the binding site.

In another embodiment, the method of drug design involves computer-assisted design of chemical entities that associate with Hepatitis C virus helicase, its homologs, or portions thereof. Chemical entities can be designed in a step-wise fashion, one fragment at a time, or may be designed as a whole or “de novo.”

To be a viable drug candidate, the chemical entity identified or designed according to the method must be capable of structurally associating with at least part of a Hepatitis C virus helicase or Hepatitis C virus helicase-like binding sites, and must be able, sterically and energetically, to assume a conformation that allows it to associate with the Hepatitis C virus helicase or Hepatitis C virus helicase-like binding site. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the binding site, and the spacing between various functional groups of an entity that directly interact with the Hepatitis C virus helicase-like binding site or homologs thereof.

Optionally, the potential binding of a chemical entity to a Hepatitis C virus helicase or Hepatitis C virus helicase-like binding site is analyzed using computer modeling techniques prior to the actual synthesis and testing of the chemical entity. If these computational experiments suggest insufficient interaction and association between it and the Hepatitis C virus helicase or Hepatitis C virus helicase-like binding site, testing of the entity is obviated. However, if computer modeling indicates a strong interaction, the molecule may then be synthesized and tested for its ability to bind to or interfere with a Hepatitis C virus helicase or Hepatitis C virus helicase-like binding site. Binding assays to determine if a compound actually binds to Hepatitis C virus helicase can also be performed and are well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One skilled in the art may use one of several methods to screen chemical entities or fragments for their ability to associate with a Hepatitis C virus helicase or Hepatitis C virus helicase-like binding site. This process may begin by visual inspection of, for example, a Hepatitis C virus helicase or Hepatitis C virus helicase-like binding site on the computer screen based on the Hepatitis C virus helicase structure coordinates in Tables 1, 2, or 3 or other coordinates which define a similar shape generated from the machine-readable storage medium. Selected fragments or chemical entities may then be positioned in a variety of orientations, or docked, within the binding site. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID (Goodford, J. Med. Chem., 28:849-57 (1985); available from Oxford University, Oxford, UK); MCSS (Miranker et al., Proteins: Struct. Funct. Gen. , 11:29-34 (1991); available from Molecular Simulations, San Diego, Calif.); AUTODOCK (Goodsell et al., Proteins: Struct. Funct. Genet., 8:195-202 (1990); available from Scripps Research Institute, La Jolla, Calif.); and DOCK (Kuntz et al., J. Mol. Biol., 161:269-88 (1982); available from University of California, San Francisco, Calif.).

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of Hepatitis C virus helicase. This would be followed by manual model building using software such as QUANTA or SYBYL (Tripos Associates, St. Louis, Mo.).

Useful programs to aid one of skill in the art in connecting the individual chemical entities or fragments include, without limitation, CAVEAT (P. A. Bartlett et al., in Molecular Recognition in Chemical and Biological Problems, Special Publ., Royal Chem. Soc., 78:182-96 (1989); Lauri et al., J. Comput. Aided Mol. Des., 8:51-66 (1994); available from the University of California, Berkeley, Calif.); 3D database systems such as ISIS (available from MDL Information Systems, San Leandro, Calif.; reviewed in Martin, J. Med. Chem. 35:2145-54 (1992)); and HOOK (Eisen et al., Proteins: Struc., Funct., Genet., 19:199-221 (1994); available from Molecular Simulations, San Diego, Calif.).

Hepatitis C virus helicase binding compounds may be designed “de novo” using either an empty binding site or optionally including some portion(s) of a known inhibitor(s). There are many de novo ligand design methods including, without limitation, LUDI (Bohm, J. Comp. Aid. Molec. Design., 6:61-78 (1992); available from Molecular Simulations Inc., San Diego, Calif.); LEGEND (Nishibata et al., Tetrahedron, 47:8985 (1991); available from Molecular Simulations Inc., San Diego, Calif.); LeapFrog (available from Tripos Associates, St. Louis, Mo.); and SPROUT (Gillet et al., J. Comput. Aided Mol. Design, 7:127-53 (1993); available from the University of Leeds, UK).

Once a compound has been designed or selected by the above methods, the efficiency with which that entity may bind to or interfere with a Hepatitis C virus helicase or Hepatitis C virus helicase-like binding site may be tested and optimized by computational evaluation. For example, an effective Hepatitis C virus helicase or Hepatitis C virus helicase-like binding site inhibitor must preferably demonstrate a relatively small difference in energy between its bound and free states (i.e., a small deformation energy of binding). Thus, the most efficient Hepatitis C virus helicase or Hepatitis C virus helicase-like binding site inhibitors should preferably be designed with a deformation energy of binding of not greater than about 10 kcal/mole; more preferably, not greater than 7 kcal/mole. Hepatitis C virus helicase or Hepatitis C virus helicase-like binding site inhibitors may interact with the binding site in more than one conformation that is similar in overall binding energy. In those cases, the deformation energy of binding is taken to be the difference between the energy of the free entity and the average energy of the conformations observed when the inhibitor binds to the protein.

An entity designed or selected as binding to or interfering with a Hepatitis C virus helicase or Hepatitis C virus helicase-like binding site may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. (1995)); AMBER, version 4.1 (P. A. Kollman, University of California at San Francisco, (1995)); QUANTA/CHARMM (Molecular Simulations, Inc., San Diego, Calif. (1995)); Insight II/Discover (Molecular Simulations, Inc., San Diego, Calif. (1995)); DelPhi (Molecular Simulations, Inc., San Diego, Calif. (1995)); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach encompassed by this invention is the computational screening of small molecule databases for chemical entities or compounds that can bind in whole, or in part, to a Hepatitis C virus helicase or Hepatitis C virus helicase-like binding site. In this screening, the quality of fit of such entities to the binding site may be judged either by shape complementarity or by estimated interaction energy (Meng et al., J. Comp. Chem., 13:505-24 (1992)).

Yet another approach to rational drug design involves an iterative process to identify inhibitors of HCV helicase. Iterative drug design is a method for optimizing associations between a protein and a compound by determining and evaluating the three-dimensional structures of successive sets of protein/compound complexes. In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three dimensional structure of the complex, and comparing the associations between the new protein/compound complex and previously solved protein/compound complexes. By observing how changes in the compound affected the protein/compound associations, these associations may be optimized.

Pharmaceutical Compositions

Pharmaceutical compositions of this invention comprise an inhibitor of Hepatitis C virus helicase activity identified according to the invention, or a pharmaceutically acceptable salt thereof, and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof. Optionally, the pH of the formulation is adjusted with pharmaceutically acceptable acids, bases, or buffers to enhance the stability of the formulated compound or its delivery form.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. Oral administration or administration by injection is preferred. The term parenteral as used herein includes subcutaneous, intracutaneous, intravenous, intramuscular, intra-articular, intrasynovial, intrastemal, intrathecal, intralesional, and intracranial injection or infusion techniques.

Dosage levels of between about 0.01 and about 100 mg/kg body weight per day, preferably between about 0.5 and about 75 mg/kg body weight per day of the Hepatitis C virus helicase inhibitory compounds described herein are useful for the prevention and treatment of Hepatitis C virus helicase mediated disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

In order that this invention be more fully understood, the following examples are set forth. These examples are for the purpose of illustration only and are not to be construed as limiting the scope of the invention in any way.

### EXAMPLES

### Example 1

Crystal Preparation and Data Collection

Materials and Methods

The HCV helicase is expressed in the yeast S. cerevisiae. The plasmid pd.hel1 was constructed by ligating the following fragments: a) a 1365 bp BamHI-HindIII fragment from plasmid pPGAP/AG containing the ADH2/GAPDH promoter sequences; b) a 1424 bp HindIII-SalI fragment generated by PCR, encoding HCV-1 helicase (aa 1193-1658) followed by a stop codon; and c) a 13.1 kbp BamHI-SalI fragment from the yeast/bacterial shuttle vector pBS24.1 which contains pBR322 sequences, leu2 gene, URA3 gene, 2-micron sequences, and the alpha-factor terminator. The plasmid pd.hel1.His was constructed from pd.hel1 by the addition of oligonucleotides which created 6 histidine codons at the 3′ end of the helicase sequence.

The plasmid pd.hel1.His was transformed into S. cerevisiae strain AD3 (Chiron) using a lithium acetate protocol. Transformants were selected on ura-plates. Single colonies were patched onto leu-plates. Starter cultures were grown in leu-8% glucose media and then inoculated into YEPD (about 1% yeast extract; about 2% peptone; and about 2% glucose). The induction of helicase expression occurs after the glucose is depleted from the YEPD media.

Purification

Yeast cells (160 gram) were broken using the Dynomill in the following buffer: 50 mM TRIS HCl pH 8.0/0.1M NaCl/0.1% octyl-glucoside. The lysate was centrifuged 25000× g for 1 hour and the pellet discarded. The supernatant was diluted with 25mM TRIS HCl pH 8.0; 0.1% octyl-glucoside to a conductivity below 3.3 mS/cm. A 1 liter TMAE Fractogel column was equilibrated with 50 mM TRIS HCl pH 8.0; 0.1% octyl-glucoside. The supernatant was loaded onto the column at a linear flow rate of 39 cm/hr. The column was washed to baseline with equilibration buffer. The protein was eluted with a 10 column volume gradient from 0 M NaCl to 0.3 M NaCl in equilibration buffer. A Ni Chelating Sepharose Fast Flow column was equilibrated with 20 mM TRIS Cl pH 7.9 0.5M NaCl 5 mM Imidazole 0.1% octyl-glucoside. Five hundred mM NaCl and 5 mM imidazole was added to the TMAE pool and loaded onto the column at a linear flow rate of 238 cm/hr. The column was washed to baseline with equilibration buffer made 60 mM Imidazole. The column was eluted with a 15 column volume gradient from 60 mM to 350 mM Imidazole in equilibration buffer. The HCV Helicase was exchanged into the following buffer: 50 mM TRIS-HCl pH 8.0/0.5M NaCl/10% Glycerol/0.1% octyl-glucoside/5 mM BME. The concentration and buffer exchange was done using an Amicon stirred cell with 30K cut off membrane. The complete sequence of the HCV-1 genotype 1a construct used is shown in FIG. 2.

Crystallization

Crystals having a morphology of tetragonal bipyramids were grown by vapor diffusion with extensive macro-seeding from precipitant solutions of 6-12% PEG 5000MME (Fluka, Sigma-Aldrich Co., Inc., Milwaukee, Wis.) and 10 mM HEPES pH 7.5 (Sigma, Sigma-Aldrich Co., Inc., Milwaukee, Wis.). Orthorhombic crystals were grown from 3.0-7% PEG4000; 10% DMSO; 0.06M K₂PO₄ solutions by vapor diffusion utilizing extensive macro-seeding.

An HCV helicase crystal grown from 12.08 mg/ml protein in 10 mM Na HEPES pH 7.5; 1 mM EDTA; 5 mM DTT with 8% PEG5000 MME on a sitting drop bridge was mounted at room temperature in a glass capillary for diffraction data collection. The crystal was about 0.12×0.12×0.05 mm in size. The data were collected on the single Siemens Hi-Star proportional counter mounted on the two-theta arm of a Siemens four-circle goniostat, positioned 14 cm from the crystal at an angle of 10° from the incident beam (Brüker AXS, Madison, Wis.). A Siemens rotating anode X-Ray generator operated at 5.0 kW and equipped with graphite monochromator served as the source of CuKα X-Rays. Data were collected in four 60° scans through omega, with each image recording intensities through a 0.25° rotation. This data set is identified as “ux0723”. Data were integrated and scaled with XENGEN v2.1 software (Howard et al., J. Appl. Cryst., 20:383-87 (1987)). Observed unit cell parameters for this and other crystals described herein are shown in Table 4, along with other parameters that are measures of the quality of the diffraction data. This data was used for initial molecular replacement toward the solution of this structure, but was later superceded by higher resolution data collected at beamline 17-ID of the Advanced Photon Source (APS) at Argonne National Labs (aps026).

A superior HCV helicase crystal was grown from 3 microliters of 9.13 mg/ml protein in 10 mM Na HEPES 7.5; 1 mM EDTA; 5 mM DTT mixed with 3 microliters of 14% PEG5000 MME on a sitting drop bridge seeded with a dilute microseed stock (after 1 hour pre-equilibration). The crystal appeared after about 1 week. A final size of about 0.4×0.4×0.2 mm was observed. The crystal was transferred into a cryogenic solution [0.8 ml of (10% glycerol, 10% PEG5000 MME, 100 mM Na HEPES pH 7.5) mixed with 0.2 ml glycerol] and equilibrated for 35 minutes. The crystal was then plunged into liquid propane and the frozen crystal transferred to APS in liquid nitrogen for data collection. The synchrotron data were collected with the sample under a dry liquid-N₂-controlled cold stream (Oxford CryoSystems) at 100K. Incident radiation with wavelength λ=1.0000 Å was used. All data were collected in a single 100° omega scan in 0.25° increments, and recorded on a Brüker CCD detector operated in binned (1 k) mode. The crystal-to-detector distance was 15.0 cm. Two-dimensional images were integrated and scaled with the SAINT (v 5) software system as implemented by staff of the Industrial Macromolecular Crystallography Association (IMCA). This crystal yielded 2.0 Å diffraction data used in structural refinement of this crystal form. This data is identified as “aps026” in Table 4.

Diffraction data from two crystals were used in the structure analysis. The crystal that ultimately gave rise to diffraction data identified as “ux0770” was transferred to successive 10 microliter drops containing increasing concentrations of cryogenic solution (0.06M potassium phosphate; 7% PEG8000; 10% DMSO; 25% Glycerol). Two minutes in 1 microliter cryogenic solution +9 microliters well mix; two minutes in 2 microliters cryogenic solution +8 microliters well mix; two minutes in 4 microliters cryogenic solution +6 microliters well mix; two minutes in 6 microliters cryogenic solution +4 microliters well mix; two minutes in 8 microliters cryogenic solution +2 microliters well mix; and 10 microliters cryogenic solution for two minutes. The crystal was frozen in liquid nitrogen in a Hampton fiber loop and maintained at 100 K under a dry liquid-N₂-controlled cold stream (Oxford Cryosystems, Oxford, U.K.) during diffraction data collection. Diffraction data were collected on the Single Hi-Star detector (Siemens) at a crystal-to-detector distance of 14 cm and 2 theta angle was 20°. In this configuration, the detector can acquire 2.2 Å resolution data. Data were collected in 0.25° increments in seven 60° omega scans. The crystal was somewhat mosaic but gave very good diffraction. Data were integrated and scaled with XENGEN v.2.1 software (Howard et al., J. Appl. Cryst., 20:383-87 (1987)).

Another crystal that ultimately gave rise to diffraction data identified as “ux0771” was transferred to successive 10 microliter drops containing increasing concentrations of cryogenic solution and frozen as described above. Data were collected at 100K on a Siemens Dual Hi-Star detector system mounted on a rotating anode X-ray source equipped with Gobel mirrors. The master detector was placed at a 2 theta angle 35° from the incident beam at distance of 15 cm from the crystal. The second (slave) detector is then at an effective 2 theta of −12.84° to intercept low resolution data. Diffraction data were measured to 1.8 Å (Table 4).

TABLE 4
Diffraction data summary

| | Tetragonal Form | | Orthorhombic Form | |
|---|---|---|---|---|
| Space Group | P4₁ | | P2₁2₁2 | |
| Data set ID | ux0723 | aps026 | ux0770 | ux0771 |
| Cell Parameters | | | | |
| a | 112.34 Å | 109.57 Å | 66.14 Å | 66.21 Å |
| b | 112.34 Å | 109.57 Å | 109.86 Å | 110.12 Å |
| c | 87.37 Å | 84.08 Å | 63.87 Å | 64.21 Å |
| Resolution | 3.4 Å | 2.0 Å | 2.3 Å | 1.8 Å |
| No. Observations | 31,652 | 302,188 | 104,158 | 167,634 |
| No. Unique reflections | 11,173 | 64,446 | 21,598 | 43,264 |
| % Completeness | 73% | 96% | 99% | 96% |
| R_{sym} | 0.097 | 0.082 | 0.043 | 0.055 |

### Example 2

X-ray Crystal Structure Solutions Tetragonal Form

Crystals were assigned to one of the two enantiomorphic space groups P4₁ or P4₃ on the basis of scaling statistics and systematic absences in the diffraction data. The final choice (P4₁) was made on the basis of superior translation function results (described below). The volume of the asymmetric unit is large enough to accommodate two independent helicase molecules, although no particular noncrystallographic symmetry was evident from inspection of self-rotation functions with GLRF (Tong et al., Acta Crystallogr., A46:783-92 (1990)).

The structure was solved by application of molecular replacement methods as implemented in X-Plor v3.851 (Brünger, X-PLOR Manual. Version 3.1: A system for crystallography and NMR, New Haven, Yale University Press (1992)). An initial search model was constructed using atomic coordinates from an HCV genotype 1b structure, which were later deposited in the Protein Data Bank as entry 8OHM (Cho et al., J. Biol. Chem., 273:15045-52 (1998)). A rotation function was computed and peaks subjected to PC-refinement (Brünger, Acta Crystallogr., A46:46-57 (1990)) using 4-8 Å diffraction data with F_{obs}≧4σ_{F}. The first rotation function searches and PC-refinement calculations conduced with this model (containing all three domains) yielded very poor Patterson correlations (0.03-0.05). In retrospect, as the “closed” form 8OHM bears the least resemblance to the “open” form UHCV, it is apparent why the initial attempts to use the 8OHM model for molecular replacement failed.

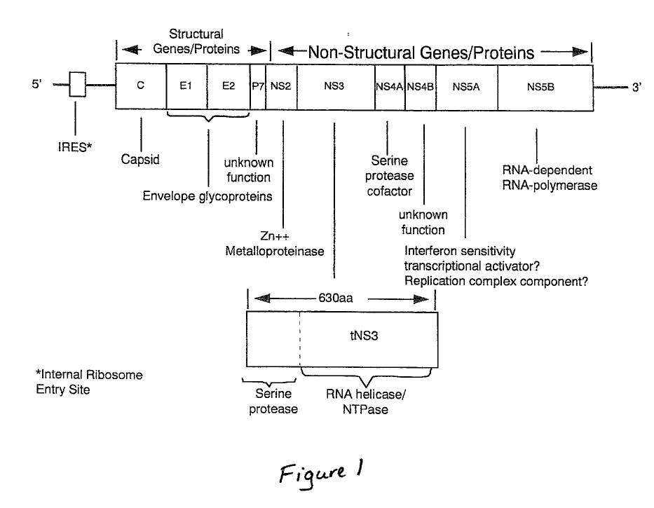

A second search model was constructed by applying the stereo figure reconstruction algorithm of Rossmann (formerly available from the Protein Data Bank) to FIG. 1 of Yao et al. (Nat. Struct. Biol., 4: 463-77 (1997)) to deduce approximate coordinates for the 1HEI structure. The coordinates derived from this procedure were prone to large errors, but they were eventually sufficient to reveal a 15-20° difference in the position of domain 2 relative to 1HEI. A series of different models based on the initial coordinates of 8OHM that sampled a range of motions of the hinge at 5° increments spanning the distribution represented by the two available structures (8OHM and our modeled version of 1HEI) was constructed. These were subsequently used as a battery of different search models in molecular replacement. None of these models gave a statistically convincing solution. In hindsight, however, it was apparent that the range of hinge motions generated was not large enough to include the correct solution. Nevertheless, the comparison of peak lists from many of the different hinge models helped identify two rotations that were present in search results with a number of different models. In addition, a subsequent search with a truncated model from which domain 2 had been removed altogether gave peaks in Patterson correlation filtering that were comparable to those found with any of the hinge models. This truncated model contained only domains 1 and 3 (residues 181-325 and 484-624). While the heights of these peaks were low (PC=0.045-0.050), their persistence in rotation functions with several different models made them stand out. Subsequent translation function searches carried out with any of the hinge models resulted in the same solution, but R-values varied, and the model with domain 2 removed gave the best statistics.

Two persistent and independent rotations identified by PC-filtering (with PC=0.0496 and 0.0449, respectively) were carried through the X-Plor translation functions, where two convincing solutions for both molecular positions (9σ above background) were identified only for enantiomorphic space group P4₁. This model consisting of two molecules (each with only two domains) resulted in an R-value of 0.405. Electron density computed with this model was sufficiently interpretable to allow approximate placement of the missing domain 2 (residues 326-483) of one molecule. A new model including all three domains was then reinserted as the search probe through all molecular replacement calculations, yielding universally more convincing results. The R-value after final rigid body refinement with both complete molecules was 0.343.

All subsequent work was conducted with synchrotron data set aps026. The molecular replacement procedures were repeated as described above using the last complete model. This search against new data produced equivalent results, but with significantly better statistics. Refinement was initiated with X-Plor positional refinement, followed by a single round of X-Plor simulated-annealing refinement (Brünger, J. Mol. Biol., 203:803-16 (1988)), using scripts generated automatically by Quanta (Molecular Simulations Inc.) for this purpose. The R-value was reduced to 0.269 for 10-2.0 Å data, while the R_{free} (cross-validation R based on an 8% random reflection sampling; Brünger, J. Mol. Biol., 203:803-16 (1988)) was reduced from 0.410 to 0.353. Subsequent refinement was completed with constrained least-squares of PROFFT (Hendrickson et al., “Incorporation of Stereochemical Information into Crystallographic Refinement” in Computing in Crystallography, (Diamond, R., Ramaseshan, S. and Ventkatesan, K. eds.), Indian Academy of Sci, Bangalore, India. pp.13.01-13.25 (1980); Finzel, J. Appl. Cryst., 20:53-55 (1987)) interspersed with frequent model analysis, map interpretation and rebuilding conducted with the CHAIN (v7.1) modeling package (Sack, J. Mol. Graphics, 6:224-25 (1988)). During this rebuilding, the amino acid sequence represented by the model was corrected to reflect the HCV-1 strain 1a sequence of FIG. 2. Final agreement factors and model geometry measures are summarized in Table 5.

Orthorhombic Form

Crystals were assigned to space group P2₁2₁2 based on scaling statistics and systematic absences in the diffraction data. The asymmetric unit contained only one helicase molecule (Vₘ=2.3) (Matthews, J. Mol. Biol., 33:491-97 (1968)). Molecular replacement was first attempted with X-Plor using the refined tetragonal form model (Molecule A) consisting of only domains 1 and 3. A single prominent rotation with PC=0.135 was readily identified from a rotation function analysis of 4-8 Å data of ux0770. A translation solution 8σ above background was also identified. This model resulted in an R-value of 0.45 (8-3 Å). Molecular replacement was repeated with this same model using the AMoRe program (Navaza, J. Acta Crystallogr., A50:157-63 (1994)) of the CCP4 package (Collaborative Computational Project Number 4, Acta Cryst., D50:760-63 (1994)), which gave an equivalent solution (but much more quickly).

An examination of molecular packing implicit in this solution led us to conclude that domain 2 could only be accommodated in an orientation roughly equivalent to its position in the tetragonal form crystals. Domain 2 was fit to poor density calculated from the AMoRe model, and the rotation/translation search repeated in AMoRe, resulting in a model with R=0.41 (8-4 Å). The position of domains 1/3 and 2 were refined as rigid bodies with X-Plor, and then the model was subjected to constrained least-squares refinement of all positional parameters by PROFFT. Electron density defining large segments of domain 2 (initially examined at 2.3 Å resolution) was initially poor, but was clarified somewhat by computation of “omit” maps in which all of domain 2 omitted from the model. Individual segments of domain 2 were repositioned manually as suggested by density throughout the refinement process, which was long and tedious, but gradually electron density maps improved. Diffraction data was superceded by higher resolution data of ux0771 following PROFFT cycle 32, and all 1.8 Å data was gradually included in the refinement. The final R-value is 0.206. Final agreement factors and model geometry measures are summarized in Table 5.

TABLE 5
Refinement statistics

| | Tetragonal Form | Orthorhombic |
|---|---|---|
| Form | aps026 | ux0771 |
| Final Reflection agreement | | |
| Resolution of data used | 10.0 B 2.0 Å | 6.0 B 1.8 |
| Å | (F_{obs} ≧ 4σ_{F}) | (F_{obs} ≧ 4σ_{F}) |
| Final R-value | 0.228 | 0.206 |
| No. of reflections used | 55,087 | 33,899 |
| Final model characteristics | | |
| Protein atoms | 6,578 | 3210 |
| Solvent atoms | 367 | 306 |
| Mean Isotropic B | 22.9 | 16.5 |
| Model geometry conformity | Rins deviation from ideality | |
| | (Target σ) | |
| Distances (Å) | | |
| 1-2 (Bond) | 0.023 (0.030) | 0.019 (0.030) |
| 1-3 (Bond angle) | 0.038 (0.040) | 0.031 (0.040) |
| 1-4 (Fixed torsion angle) | 0.041 (0.050) | 0.030 (0.050) |
| Planes (Å) | | |
| Peptides | 0.017 (0.030) | 0.015 (0.030) |
| Other | 0.021 (0.030) | 0.015 (0.030) |
| Chiral Volumes (Å³) | 0.300 (0.300) | 0.197 (0.250) |
| Non-bonded contacts (Å) | | |
| 1-4 | 0.189 (0.400) | 0.172 (0.300) |
| Possible H-bonds | 0.248 (0.400) | 0.176 (0.300) |
| Other | 0.218 (0.400) | 0.180 (0.300) |
| Thermal Parameters (Mean ΔB; Å²) | | |
| 1-2 (Main-chain atoms) | 0.957 (2.000) | 1.200 (3.000) |
| 1-2 (side-chain atoms) | 1.043 (1.500) | 1.266 (2.000) |
| 1-3 | 1.625 (3.000) | 1.909 (4.000) |

### Example 3

Comparison of HCV Helicase Structures

Coordinates for 1HEI-A, 1HEI-B, 1AIV and 8OHM were obtained from the Protein Data Bank. Atomic coordinates from different structures (including UHCV-A, UHCV-B and UHHO as described herein) were overlaid (Table 6) using a program that forces a superposition of all common atoms to requested pairs of residues in two structures using the methods of Kabsch (Acta Cryst., A34:827-28 (1978)). Because the tetragonal form (UHCV-A) appears to represent an extreme closed conformation for domain 2, this model was chosen as the resting state for comparison to other geometries. The root mean square distances given in Table 6 reflect the distances between alpha carbons following superposition of all atoms common to both structures.

TABLE 6
R.M.S. difference in alpha-carbon positions (Å) after superposition
of helicase coordinates from different crystal forms

| Structure^{H} | Fragments^{I} | UHCV-B | UHHO | 1HEI-A | 1HEI-B | 1A1V | 8OHM |
|---|---|---|---|---|---|---|---|
| UHCV-A | d1 vs d1 | 1.13 | 1.02 | 1.15 | 1.41 | 1.47 | 0.98 |
| | d2 vs d2 | 0.57 | 0.93 | 1.36 | 1.58 | 0.77 | 0.89 |
| | d3 vs d3 | 0.50 | 0.57 | 0.85 | 0.90 | 0.38 | 0.68 |
| | d1/d3 vs d1/d3 | 0.90 | 0.90 | 1.02 | 1.20 | 1.08 | 0.87 |
| | all vs all | 0.89 | 1.52 | 2.08 | 2.55 | 1.52 | 4.18 |
| UHCV-B | d1 vs d1 | — | 0.46 | 0.55 | 0.82 | 1.00 | 0.64 |
| | d2 vs d2 | — | 0.85 | 1.35 | 1.60 | 0.84 | 0.90 |
| | d3 vs d3 | — | 0.40 | 0.93 | 0.95 | 0.54 | 0.56 |
| | d1/d3 vs d1/d3 | — | 0.55 | 0.85 | 0.98 | 0.88 | 0.67 |
| | all vs all | — | 1.34 | 2.17 | 2.65 | 1.60 | 4.28 |
| UHHO | d1 vs d1 | — | — | 0.59 | 0.89 | 0.98 | 0.65 |
| | d2 vs d2 | — | — | 1.53 | 1.79 | 1.08 | 1.02 |
| | d3 vs d3 | — | — | 0.96 | 0.99 | 0.52 | 0.62 |
| | d1/d3 vs d1/d3 | — | — | 0.91 | 1.09 | 0.82 | 0.69 |
| | all vs all | — | — | 2.06 | 2.58 | 1.53 | 3.61 |
| 1HEI-A | d1 vs d1 | — | — | — | 0.82 | 0.90 | 0.79 |
| | d2 vs d2 | — | — | — | 1.16 | 1.00 | 1.01 |
| | d3 vs d3 | — | — | — | 0.44 | 0.91 | 0.98 |
| | d1/d3 vs d1/d3 | — | — | — | 0.67 | 0.93 | 0.91 |
| | all vs all | — | — | — | 1.23 | 1.13 | 2.67 |
| 1A1V | d1 vs d1 | — | — | — | — | — | 1.17 |
| | d2 vs d2 | — | — | — | — | — | 0.66 |
| | d3 vs d3 | — | — | — | — | — | 0.73 |
| | d1/d3 vs d1/d3 | — | — | — | — | — | 0.99 |
| | all vs all | — | — | — | — | — | 3.11 |

^{H}Structures

| | |
|---|---|
| UHCV-A | Tetragonal form molecule A (described herein) |
| UHCV-B | Tetragonal form molecule B (described herein) |
| | (space group P4₁; a = b = 109.57 Å, c = 84.08 Å; Z = 2) |
| UHHO | Orthorhombic form (described herein); |
| | 360-361, 393-396 missing |
| | (space group P2₁2₁2; a = 66.14 Å, b = 109.57 Å, |
| | c = 63.87 Å; Z = 1) |
| 1HEI-A | Schering-Plough (S-P) orthorhombic form molecule A |
| | (Yao et al., Nat. Struct. Biol., 4: 463-77 (1997)) |
| 1HEI-B | S-P orthorhombic form molecule B; 233-261 missing |
| | (Yao et al., Nat. Struct. Biol., 4: 463-77 (1997); |
| | space group P2₁2₁2₁; a = 81.54 Å, b = 102.73 Å, |
| | c = 119.50 Å; Z = 2) |
| 8OHM | Pohang trigonal form; 417-420 missing |
| | (Cho et al., J. Biol. Chem., 273:15045-52 (1998); |
| | space group P3₁21; a = b = 93.3 Å, c = 104.6 Å) |
| 1A1V | Vertex orthorhombic form; 415-417 missing |
| | (Kim et al., Structure, |
| | 6: 89-100 (1998); space group P2₁2₁2; a = 73.10 Å, |
| | b = 117.50 Å, c = 63.40 Å) |

^{I}Fragments

| | |
|---|---|
| d1 | Domain 1; residues 192-326. |
| d2 | Domain 2; residues 327-483, excluding hairpin 435-446. |
| d3 | Domain 3; residues 484-624. |

The movement of domain 2 results in a change in teh distance separating oligonucleotide binding sites in domain 1 and domain 2. The distance between these sites is defined as the distance between the side chain oxygen of T₂₆₉ and the side chain oxygen of T₄₁₁ and is tabulated for each structure in Table 7.

TABLE 7
Distances

| | UHCV-A | UHHO | 1A1V | 1HEI-A | 8OHM |
|---|---|---|---|---|---|
| | (this report) | (this report) | (Vertex) | (S-P) | (Pohang) |
| Distance | 19.3 Å | 19.2 Å | 21.5 Å | 22.3 Å | 26.8 Å |
| between | | | | | |
| DNA | | | | | |
| binding | | | | | |
| motifs | | | | | |
| of domain 1 | | | | | |
| and 2 | | | | | |

### Example 4

Preparation of a Helicase/Ligand Complex (Tetragonal Form)

To prepare a co-crystalline complex of HCV helicase with a chemical entity, native tetrahedral crystals (UHCV) grown as described in Example 1 were transferred into a cryogenic solution [10% (v/v) glycerol, 10% PEG5000MME, 10 mM HEPES (pH 7.5)] and stabilized overnight. This crystal was soaked in 88% cryogenic solution, 1 mM MgCl₂ and 10 mM of the chemical entity for 2 days prior to being frozen in liquid nitrogen for data collection. The crystals had a dark orange appearance. Diffraction data were obtained at the Advanced Photon Source beam-line 17-ID as described in Example 1. Crystals diffracted to 2.0 Å. Analysis of this data revealed that the chemical entity is bound spanning the oligonucleotide binding sites, making interactions with G₂₅₅ and T₂₆₉ on domain 1; and R₃₉₃ and T₄₁₁ on domain 2.

Similar treatment of orthorhombic form crystals (UHHO) with the same chemical entity resulted in no apparent formation of complex. This may be the result of the incorrect (suboptimal) spacing separating oligonucleotide-binding sites in the orthorhombic crystal form. The same result may be expected with other alternate crystal forms of HCV helicase. This result demonstrates the unique utility of the tetrahedral (UHCV) crystal form for studying some ligands that bind spanning the oligonucleotide binding sites.

### Example 5

Preparation of a Helicase/Ligand Complex (Orthorhombic Form)

To prepare a co-crystalline complex of HCV helicase with another ligand, native orthorhombic crystals (UHHO) grown as described in Example 1 were transferred into a stabilization solution of 7% PEG4000, 5% DMSO, and 10 mM ligand. After a few hours the crystals were sequentially transferred (30 minutes each soak) into stabilization solutions containing progressively higher DMSO concentrations. The final concentration reached was 20% DMSO. The crystal was then frozen in liquid nitrogen. Diffraction data was obtained and analyzed as described in Example 1. Crystals diffracted to 1.8 Å. Analysis of this data revealed that the ligand is bound in the NTP-binding site of HCV helicase.

Similar treatment of tetragonal form crystals (UHCV) with the same compound resulted in visible cracking of the crystals, and a complete loss of diffraction. This result demonstrates the unique utility of the orthorhombic crystal form (UHHO) for studying ligands that bind at the NTP-binding site, and the possible unsuitability of the tetragonal crystals for this purpose.

The complete disclosure of all patents, patent applications including provisional applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described; many variations will be apparent to one skilled in the art and are intended to be included within the invention defined by the claims.

Sequence Listing Free Text

SEQ ID NO: 1 Hepatitis C virus (HCV) NS3 helicase

SEQ ID NO: 2 Conserved NTP-binding loop (Walker motif A) in Hepatitis C virus (HCV) NS3 helicase

SEQ ID NO: 3 Conserved sequence motif VI in Hepatitis C virus (HCV) NS3 helicase

What is claimed is:

1. A molecule or molecular complex comprising at least a portion of a Hepatitis C virus helicase or Hepatitis C virus helicase-like domain 1/domain 2 interface, wherein the domain 1/domain 2 interface comprises amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively.

2. A molecule or molecular complex comprising at least a portion of a Hepatitis C virus helicase or Hepatitis C virus helicase-like oligonucleotide binding site, wherein the oligonucleotide binding site comprises amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively.

3. A Hepatitis C virus helicase molecule or molecular complex comprising at least a first and a second oligonucleotide binding site, wherein the distance between the first and the second oligonucleotide binding sites is less than about 21 angstroms.

4. The Hepatitis C virus helicase molecule or molecular complex of claim 3, wherein the distance between the first and the second oligonucleotide binding sites is about 18.8 to about 19.5 angstroms.

5. A molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3.

6. A scalable three-dimensional configuration of points, at least a portion of said points derived from structure coordinates of at least a portion of a Hepatitis C virus helicase molecule or molecular complex as listed in Tables 1, 2, or 3 and comprising at least one of a Hepatitis C virus helicase or Hepatitis C virus helicase-like domain 1/domain 2 interface, domain 1 oligonucleotide binding site, or domain 2 oligonucleotide binding site.

7. The scalable three-dimensional configuration of points of claim 6, wherein substantially all of said points are derived from structure coordinates of a Hepatitis C virus helicase molecule or molecular complex as listed in Tables 1, 2, or 3.

8. The scalable three-dimensional configuration of points of claim 6 wherein at least a portion of the points derived from the Hepatitis C virus helicase structure coordinates are derived from structure coordinates representing the locations of at least the backbone atoms of amino acids selected from the group consisting of (1) domain 1/domain 2 interface amino acids 205-209, 232-238, 415-420 and 460-467, (2) domain 1 oligonucleotide binding site amino acids 230-232, 255, 269, and 270-272, and (3) domain 2 oligonucleotide binding site amino acids 391-393, 411-413, 415, 416 and 460; as represented by structure coordinates of UHCV-A, UHCV-B, or UHHO in Tables 1, 2, and 3 respectively.

9. The scalable three-dimensional configuration of points of claim 6 displayed as a holographic image, a stereodiagram, a model or a computer-displayed image.

10. A scalable three-dimensional configuration of points, at least a portion of the points derived from structure coordinates of at least a portion of a molecule or a molecular complex that is structurally homologous to a Hepatitis C virus helicase molecule or molecular complex and comprises at least one of a Hepatitis C virus helicase or Hepatitis C virus helicase-like domain 1/domain 2 interface, domain 1 oligonucleotide binding site, or domain 2 oligonucleotide binding site.

11. The scalable three-dimensional configuration of points of claim 10 displayed as a holographic image, a stereodiagram, a model or a computer-displayed image.

12. A machine-readable data storage medium comprising a data storage material encoded with machine readable data which, when using a machine programmed with instructions for using said data, is capable of displaying a graphical three-dimensional representation of at least one molecule or molecular complex selected from the group consisting of:
(i) a molecule or molecular complex comprising at least a portion of a Hepatitis C virus helicase or Hepatitis C virus helicase-like domain 1/domain 2 interface, wherein the domain 1/domain 2 interface comprises amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(ii) a molecule or molecular complex comprising at least a portion of a Hepatitis C virus helicase or Hepatitis C virus helicase-like oligonucleotide binding site, wherein the oligonucleotide binding site comprises amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(iii) a Hepatitis C virus helicase molecule or molecular complex comprising at least a first and a second oligonucleotide binding site, wherein the distance between the first and the second oligonucleotide binding sites is less than about 21 angstroms; and
(iv) a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3.

13. A machine-readable data storage medium comprising a data storage material encoded with a first set of machine readable data which, when combined with a second set of machine readable data, using a machine programmed with instructions for using said first set of data and said second set of data, can determine at least a portion of the structure coordinates corresponding to the second set of machine readable data, wherein said first set of data comprises a Fourier transform of at least a portion of the structure coordinates for Hepatitis C virus helicase listed in Tables 1, 2, or 3; and said second set of data comprises an x-ray diffraction pattern of a molecule or molecular complex of unknown structure.

14. A method for obtaining structural information about a molecule or a molecular complex of unknown structure comprising:
crystallizing the molecule or molecular complex;
generating an x-ray diffraction pattern from the crystallized molecule or molecular complex;
applying at least a portion of the structure coordinates set forth in Tables 1, 2, or 3 to the x-ray diffraction pattern to generate a three-dimensional electron density map of at least a portion of the molecule or molecular complex whose structure is unknown.

15. A method for homology modeling a Hepatitis C virus helicase homolog comprising:
aligning the amino acid sequence of a Hepatitis C virus helicase homolog with an amino acid sequence of Hepatitis C virus helicase (SEQ ID NO: 1) and incorporating the sequence of the Hepatitis C virus helicase homolog into a model of Hepatitis C virus helicase derived from structure coordinates set forth in Tables 1, 2, or 3 to yield a preliminary model of the Hepatitis C virus helicase homolog;
subjecting the preliminary model to energy minimization to yield an energy minimized model;
remodeling regions of the energy minimized model where stereochemistry restraints are violated to yield a final model of the Hepatitis C virus helicase homolog.

16. A computer-assisted method for identifying an inhibitor of Hepatitis C virus helicase activity comprising:
supplying a computer modeling application with a set of structure coordinates for at least a portion of at least one molecule or molecular complex selected from the group consisting of:
(i) a molecule or molecular complex comprising at least a portion of a Hepatitis C virus helicase or Hepatitis C virus helicase-like domain 1/domain 2 interface, wherein the domain 1/domain 2 interface comprises amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(ii) a molecule or molecular complex comprising at least a portion of a Hepatitis C virus helicase or Hepatitis C virus helicase-like oligonucleotide binding site, wherein the oligonucleotide binding site comprises amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(iii) a Hepatitis C virus helicase molecule or molecular complex comprising at least a first and a second oligonucleotide binding site, wherein the distance between the first and the second oligonucleotide binding sites is less than about 21 angstroms; and
(iv) a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3;
wherein said portion of the molecule comprises at least one HCV binding site selected from the group consisting of an oligonucleotide binding site on domain 1, an oligonucleotide binding site on domain 2, an NTP binding site, and a domain 1/domain 2 interface;
supplying the computer modeling application with a set of structure coordinates of a chemical entity; and
determining whether the chemical entity is expected to bind to or interfere with the molecule or molecular complex.

17. The method of claim 16 wherein determining whether the chemical entity is expected to bind to or interfere with the molecule or molecular complex comprises performing a fitting operation between the chemical entity and a binding site of the molecule or molecular complex, followed by computationally analyzing the results of the fitting operation to quantify the association between the chemical entity and the binding site.

18. The method of claim 16 further comprising screening a library of chemical entities.

19. A computer-assisted method for designing an inhibitor of Hepatitis C virus helicase activity comprising:
supplying a computer modeling application with a set of structure coordinates of at least a portion of at least one molecule or molecular complex selected from the group consisting of:
(i) a molecule or molecular complex comprising at least a portion of a Hepatitis C virus helicase or Hepatitis C virus helicase-like domain 1/domain 2 interface, wherein the domain 1/domain 2 interface comprises amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(ii) a molecule or molecular complex comprising at least a portion of a Hepatitis C virus helicase or Hepatitis C virus helicase-like oligonucleotide binding site, wherein the oligonucleotide binding site comprises amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(iii) a Hepatitis C virus helicase molecule or molecular complex comprising at least a first and a second oligonucleotide binding site, wherein the distance between the first and the second oligonucleotide binding sites is less than about 21 angstroms; and
(iv) a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3;
wherein said portion of the molecule comprises at least one HCV binding site selected from the group consisting of an oligonucleotide binding site on domain 1, an oligonucleotide binding site on domain 2, an NTP binding site, and a domain 1/domain 2 interface;
supplying the computer modeling application with a set of structure coordinates for a chemical entity;
evaluating the potential binding interactions between the chemical entity and substrate binding site of the molecule or molecular complex;
structurally modifying the chemical entity to yield a set of structure coordinates for a modified chemical entity; and
determining whether the modified chemical entity is an inhibitor expected to bind to or interfere with the molecule or molecular complex, wherein binding to or interfering with the molecule or molecular complex is indicative of potential inhibition of Hepatitis C virus helicase activity.

20. The method of claim 19 wherein determining whether the modified chemical entity is an inhibitor expected to bind to or interfere with the molecule or molecular complex comprises performing a fitting operation between the chemical entity and a binding site of the molecule or molecular complex, followed by computationally analyzing the results of the fitting operation to quantify the association between the chemical entity and the binding site.

21. The method of claim 19 wherein the set of structure coordinates for the chemical entity is obtained from a chemical fragment library.

22. A computer-assisted method for designing an inhibitor of Hepatitis C virus helicase activity de novo comprising:
supplying a computer modeling application with a set of structure coordinates of at least a portion of at least one molecule or molecular complex selected from the group consisting of:
(i) a molecule or molecular complex comprising at least a portion of a Hepatitis C virus helicase or Hepatitis C virus helicase-like domain 1/domain 2 interface, wherein the domain 1/domain 2 interface comprises amino acids 205-209, 232-238, 415-420 and 460-467, the domain 1/domain 2 interface being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(ii) a molecule or molecular complex comprising at least a portion of a Hepatitis C virus helicase or Hepatitis C virus helicase-like oligonucleotide binding site, wherein the oligonucleotide binding site comprises amino acids selected from the group consisting of (1) domain 1 oligonucleotide binding site amino acids 230-232, 255, 269, and 270-272, and (2) domain 2 oligonucleotide binding site amino acids 391-393, 411-413, 415, 416 and 460; the oligonucleotide binding site being defined by a set of points having a root mean square deviation of less than about 1.5 Å from points representing the backbone atoms of said amino acids as represented by the structure coordinates of UHCV-A, UHCV-B, or UHHO as listed in Tables 1, 2, or 3 respectively;
(iii) a Hepatitis C virus helicase molecule or molecular complex comprising at least a first and a second oligonucleotide binding site, wherein the distance between the first and the second oligonucleotide binding sites is less than about 21 angstroms; and
(iv) a molecule or molecular complex that is structurally homologous to a Hepatitis C virus helicase molecule or molecular complex, wherein the Hepatitis C virus helicase molecule or molecular complex is represented by at least a portion of the structure coordinates listed in Tables 1, 2, or 3;
wherein said portion of the molecule comprises at least one HCV binding site selected from the group consisting of an oligonucleotide binding site on domain 1, an oligonucleotide binding site on domain 2, an NTP binding site, and a domain 1/domain 2 interface;
computationally building a chemical entity represented by set of structure coordinates; and
determining whether the chemical entity is an inhibitor expected to bind to or interfere with the molecule or molecular complex, wherein binding to or interfering with the molecule or molecular complex is indicative of potential inhibition of Hepatitis C virus helicase activity.

23. The method of claim 22 wherein determining whether the chemical entity is an inhibitor expected to bind to or interfere with the molecule or molecular complex comprises performing a fitting operation between the chemical entity and a binding site of the molecule or molecular complex, followed by computationally analyzing the results of the fitting operation to quantify the association between the chemical entity and the binding site.

24. The method of any of claims 16, 19, or 22 further comprising supplying or synthesizing the potential inhibitor, then assaying the potential inhibitor to determine whether it inhibits Hepatitis C virus helicase activity.

25. A method for making an inhibitor of Hepatitis C virus helicase activity, the method comprising chemically or enzymatically synthesizing a chemical entity to yield an inhibitor of Hepatitis C virus helicase activity, the chemical entity having been identified during a computer-assisted process comprising supplying a computer modeling application with a set of structure coordinates of a molecule or molecular complex, the molecule or molecular complex comprising at least a portion of at least one of a Hepatitis C virus helicase or Hepatitis C virus helicase-like binding site; supplying the computer modeling application with a set of structure coordinates of a chemical entity; and determining whether the chemical entity is expected to bind to or interfere with the molecule or molecular complex at a binding site, wherein binding to or interfering with the molecule or molecular complex is indicative of potential inhibition of Hepatitis C virus helicase activity.

26. A method for making an inhibitor of Hepatitis C virus helicase activity, the method comprising chemically or enzymatically synthesizing a chemical entity to yield an inhibitor of Hepatitis C virus helicase activity, the chemical entity having been designed during a computer-assisted process comprising supplying a computer modeling application with a set of structure coordinates of a molecule or molecular complex, the molecule or molecular complex comprising at least a portion of at least one of a Hepatitis C virus helicase or Hepatitis C virus helicase-like binding site; supplying the computer modeling application with a set of structure coordinates for a chemical entity; evaluating the potential binding interactions between the chemical entity and a binding site of the molecule or molecular complex; structurally modifying the chemical entity to yield a set of structure coordinates for a modified chemical entity; and determining whether the chemical entity is expected to bind to or interfere with the molecule or molecular complex at the binding site, wherein binding to or interfering with the molecule or molecular complex is indicative of potential inhibition of Hepatitis C virus helicase activity.

27. A method for making an inhibitor of Hepatitis C virus helicase activity, the method comprising chemically or enzymatically synthesizing a chemical entity to yield an inhibitor of Hepatitis C virus helicase activity, the chemical entity having been designed during a computer-assisted process comprising supplying a computer modeling application with a set of structure coordinates of a molecule or molecular complex, the molecule or molecular complex comprising at least a portion of at least one of a Hepatitis C virus helicase or Hepatitis C virus helicase-like binding site; computationally building a chemical entity represented by set of structure coordinates; and determining whether the chemical entity is expected to bind to or interfere with the molecule or molecular complex at a binding site, wherein binding to or interfering with the molecule or molecular complex is indicative of potential inhibition of Hepatitis C virus helicase activity.

28. An inhibitor of Hepatitis C virus helicase activity identified, designed or made according to the method of any of the claims 16, 19, 22, 25, 26, and 27.

29. A composition comprising an inhibitor of Hepatitis C virus helicase activity identified or designed according to the method of any of the claims 16, 19, 22, 25, 26, and 27.

30. A pharmaceutical composition comprising an inhibitor of Hepatitis C virus helicase activity identified or designed according to the method of any of the 16, 19, 22, 25, 26, and 27 or a salt thereof, and pharmaceutically acceptable carrier.

31. A method for crystallizing a Hepatitis C virus helicase molecule or molecular complex comprising growing a crystal from a precipitant solution comprising purified Hepatitis C virus helicase, about 3% by weight to about 14% by weight PEG, about 5% by weight to about 15% by weight DMSO, and about 0.05M to about 0.07M potassium phosphate.

32. A method for co-crystallizing a Hepatitis C virus helicase molecule and a ligand to yield a molecular complex, comprising:
exchanging purified Hepatitis C virus helicase into a solution comprising HEPES, EDTA, and dithiothreitol;
concentrating the Hepatitis C virus helicase to a concentration of about 12-16 mg/mL;
combining concentrated Hepatitis C virus helicase with the ligand in a mixture comprising about 4% by weight to about 14% by weight PEG and about 5% by weight to about 15% by weight DMSO; and
growing a co-crystal by vapor diffusion.

33. The method of claim 32 wherein combining the concentrated Hepatitis C virus helicase with the ligand in a mixture comprising PEG and DMSO and growing the co-crystal are performed in the absence of potassium phosphate.

34. The method of claim 32 wherein the ligand binds to an NTP binding site on the Hepatitis C virus helicase.

35. A method for crystallizing a Hepatitis C virus helicase molecule or molecular complex comprising growing a crystal by vapor diffusion with macro-seeding from a precipitant solution comprising purified Hepatitis C virus helicase, HEPES, and about 4% by weight to about 14% by weight mono-alkyl ether of PEG.

36. A method for co-crystallizing a Hepatitis C virus helicase molecule and a ligand to yield a molecular complex, comprising growing a crystal by vapor diffusion with macro-seeding from a precipitant solution comprising purified HCV helicase, HEPES, about 4% by weight to about 14% by weight mono-alkyl ether of PEG, and the ligand, wherein the ligand binds to at least one oligonucleotide binding site on the Hepatitis C virus helicase.

37. The method of claims 31-36 wherein the amino acid sequence of the Hepatitis C virus helicase is SEQ ID NO: 1.

38. Crystalline Hepatitis C virus helicase comprising a tetragonal crystal having unit cell dimensions of a=b=109 Å±3 Å; c=84 Å±2 Å; α=β=γ=90°; and space group P4₁; the unit cell containing two molecules in an asymmetric unit.

39. The crystalline Hepatitis C virus helicase of claim 38 wherein the amino acid sequence of Hepatitis C virus helicase is SEQ ID NO: 1.

40. Crystalline Hepatitis C virus helicase comprising an orthorhombic crystal characterized by unit cell dimensions of a=66 Å±2 Å; b=110 Å±3 Å; c=64 Å; α=β=γ=90°; and a space group P2₁2₁2; the unit cell containing one molecule in the asymmetric unit.

41. The crystalline Hepatitis C virus helicase of claim 40 wherein the amino acid sequence of Hepatitis C virus helicase is SEQ ID NO: 1.

42. Crystalline Hepatitis C virus helicase having an amino acid sequence is SEQ ID NO: 1.

43. A composition comprising crystalline Hepatitis C virus helicase of any of claims 38-42.

44. A method for solving a crystal structure of a crystal of Hepatitis C virus helicase having unit cell dimensions of a=b=109 Å±3 Å; c=84 Å±2 Å; α=β=γ=90°; and space group P4₁, the unit cell containing two molecules in an asymmetric unit, the method comprising:
generating an x-ray diffraction pattern from the crystal,
collecting diffraction data, and
analyzing the data to generate the structure coordinates for the Hepatitis C virus helicase.

45. A method for solving a crystal structure of a crystal of Hepatitis C virus helicase having unit cell dimensions of a=66 Å±2 Å; b=110 Å±3 Å; c=64 Å±2 Å; α=β=γ=90°; and a space group P2₁2₁2, the unit cell containing one molecule in an asymmetric unit, the method comprising:
generating an x-ray diffraction pattern from the crystal,
collecting diffraction data, and
analyzing the data to generate the structure coordinates for the Hepatitis C virus helicase.

46. The method of claims 44 or 45 wherein the amino acid sequence of the Hepatitis C virus helicase is SEQ ID NO: 1.

47. A method for incorporating a chemical entity in a crystal comprising placing a tetragonal crystal of Hepatitis C virus helicase having unit cell dimensions of a=b=109 Å±3 Å; c=84 Å±2 Å; α=β=γ=90°; and space group P4₁ in an aqueous solution comprising about 1 mM to about 10 mM chemical entity, and 0% by weight to about 15% by weight DMSO.

48. A method for incorporating a chemical entity in a crystal comprising placing an orthorhombic crystal of Hepatitis C virus helicase having unit cell dimensions of a=66 Å±2 Å; b=110 Å±3 Å; c=64 Å±2 Å; α=β=γ=90°; and a space group P2₁2₁2 in an aqueous solution comprising about 1 mM to about 10 mM chemical entity, and 0% by weight to about 15% by weight DMSO.
